(19) United States
(12) Patent Application Publication
(10) Pub. No.: US 2004/0191849 A1
(43) Pub. Date: Sep. 30, 2004

(54) Method for identifying fungicides

(76) Inventors: Peter Schreier, Koln (DE); Birgitta Leuthner, Langenfeld (DE); Volkhart Li, Velbert (DE); Karl-Heinz Kuck, Langenfeld (DE); Ralf Dunkel, Monheim (DE)

Correspondence Address: BAYER CROPSCIENCE LP, Patent Department, 100 BAYER ROAD, PITTSBURGH, PA, 15205-9741, US

(21) Appl. No.: 10/628,706

(22) Filed: Jul. 28, 2003

(30) Foreign Application Priority Data
Aug. 1, 2002 (DE) 10235129.5

(51) Int. Cl. C12Q 1/48; C12Q 1/18

(52) U.S. Cl. 435/15; 435/32

(57) ABSTRACT

The present invention relates to a method for identifying fungicides, to the use of ribose-5-phosphate isomerase for identifying fungicides, to the use of ribose-5-phosphate isomerase inhibitors as fungicides and to nucleic acids which encode a polypeptide with ribose-5-phosphate isomerase activity from phytopathogenic fungi.

### BACKGROUND OF THE INVENTION

The present invention relates to a method for identifying fungicides, to the use of ribose-5-phosphate isomerase for identifying fungicides, to the use of ribose-5-phosphate isomerase inhibitors as fungicides, and to nucleic acids which encode a polypeptide with ribose-5-phosphate isomerase activity from phytopathogenic fungi.

An undesired fungal growth which leads every year to considerable damage in agriculture can be controlled by the use of fungicides. The demands made on fungicides have increased constantly with regard to their activity, costs and, above all, ecological soundness. There exists therefore a demand for new substances or classes of substances which can be developed into potent and ecologically sound new fungicides. In general, it is customary to search for such new lead structures in greenhouse tests. However, such tests require a high input of labour and a high financial input. The number of substances which can be tested in the greenhouse is, accordingly, limited. An alternative to such tests is the use of what are known as high-throughput screening (HTS) methods. This involves testing a large number of individual substances with regard to their effect on cells, individual gene products or genes in an automated method. When certain substances are found to have an effect, they can be studied in conventional screening methods and, if appropriate, developed further.

Advantageous targets for fungicides are frequently searched for in essential biosynthetic pathways. Ideal fungicides are, moreover, those substances which inhibit gene products which are of decisive importance in the manifestation of the pathogenicity of a fungus. There remains a need in the art for methods for identifying new and useful targets and modulators of those targets which can be used as fungicides.

### SUMMARY OF THE INVENTION

It was therefore an object of the present invention to identify, and make available, a suitable new target for potential fungicidally active compounds and to provide a method which makes possible the identification of modulators of this target which can be used as fungicides.

It has now been found that fungal ribose-5-phosphate isomerase can be used for identifying, in suitable methods, active substances which can be employed as fungicides. Cytoplasmic ribose-5-phosphate isomerase, hereinbelow also abbreviated to “RPI”, which is also referred to as phosphopentose isomerase, phosphoriboisomerase, ribose-phosphate isomerase, 5-phosphoribose isomerase or D-ribose-5-phosphate isomerase, is the first enzyme in the nonoxidative branch of the pentose phosphate pathway (EC 5.3.1.6). In this part of the metabolism, the ratio between the pentoses and the hexoses in the organism is set, and important sugar units are converted into each other. RPI converts ribulose-5-phosphate, the product of the oxidative branch of the pentose phosphate pathway, ribose-5-phosphate, which is required as a basic unit for pyrimidine, purine, tryptophan and histidine biosynthesis (FIG. 1).

In the next step, ribose-5-phosphate is activated to phosphoribosyl pyrophosphate, which then acts as starting compound for the abovementioned metabolites and as a basic unit for what is known as the salvage pathway of the nucleic acids. In the same manner, RPI catalyzes the reaction in the opposite direction, so that ribose-5-phosphate gives ribulose-5-phosphate, which, for example, acts as substrate for phosphoribulose kinase (Calvin cycle) in the plant. Starting with ribose-5-phosphate, transketolase, transaldolase and ribulose-5-phosphate epimerase give rise, in three further steps, to the sugar units sedoheptulose-7-phosphate, glycerinaldehyde-3-phosphate, erythrose-4-phosphate and fructose-6-phosphate.

The enzyme ribose-5-phosphate isomerase has already been known for a long time. Nucleic acids encoding it have already been isolated from a large number of bacteria and from insects such as, for example, C. elegans, and plants such as, for example A. thaliana. Only very few fungal nucleic acids encoding ribose-5-phosphate isomerases have been described. In S. cerevisiae, only one RPI gene which encodes the polypeptide, namely rki1, exists, and its knock-out, that is to say inactivation, is described as being lethal (T. Miosga and F. K. Zimmermann, Curr. Genet. 30 (1996) 404-409; R. Reuter, M. Naumann, J. Bar, T. Migosa and G. Kopperschlager, Bioseparation 7(2) (1998) 107-115 ). Besides S. cerevisiae, RPI has only been described for very few other yeasts, for example for S. pombe or S. kluyveri. In contrast, no nucleic acids which encode a ribose-5-phosphate isomerase have been isolated from phytopathogenic fungi. Whether ribose-5-phosphate isomerase can be inhibited by a chemical compound in a targeted manner, and whether such an inhibition might also be achieved in vivo, i.e. in fungi as such, has been studied neither in the abovementioned yeasts nor in fungi which are pathogenic to humans or plants. Whether inhibition of ribose-5-phosphate isomerase by an active substance damages or destroys the fungus to an extent that the active substance can be used as fungicide, that is to say whether ribose-5-phosphate isomerase is suitable as target protein for fungicidal active substances, has not been studied as yet either. The fact that the knock-out of the respective gene in S. cerevisae has been classified as lethal can only be considered an indication of the suitability of the gene product encoded by it as target protein for fungicides.

It was therefore another object of the present invention to provide an RPI from a phytopathogenic fungus in order to solve the problem which has been formulated above with the aid of this polypeptide.

### BRIEF DESCRIPTION OF THE DRAWINGS

FIG. 1 illustrates the enzymatic activity of ribose-5-phosphate isomerase (RPI). D-Ribulose-5-phosphate is converted into D-ribose-5-phosphate.

FIG. 2 illustrates the heterologous expression and isolation of RPI1 from U. maydis. Lane 1: uninduced cells; lane 2: cells post-induction; lane 3: precipitate after cell disruption; lane 4: supernatant after cell disruption; lane 5: elution fraction from the Ni-column; lane 6: fraction after the PD-column; M: 10 kDa protein standard.

FIG. 3 illustrates the kinetics of the increase in the reduced nicotinamide coenzyme NADH in the triply-coupled assay system. The assay volume amounted to 35 μl. Ribose-5-phosphate was employed in a concentration of 8.3 mM. 25 ng of RPI were used. In addition, a negative control (neg. contr.), which did not contain RPI, was carried out. The formation of NADH can be monitored by the increasing relative fluorescence.

FIG. 4 is a Lineweaver-Burk plot for determining the K_{M} value. The K_{M} value for the substrate of RPI, ribose-5-phosphate, was determined by means of the increase in absorption at 290 nm, i.e. without resorting to the coupled enzymes. The measurement was carried out with 400 ng of RPI. In the diagram, 1/v is plotted in [min/mM] versus 1/[S] in [1/mM].

FIG. 5 illustrates measuring the formation of NADH as a function of the ribose-5-phosphate concentration employed (stated in the diagram in FIG. 5) when 25 ng of RPI are employed in a reaction volume of 35 μl. A concentration of 8.3 mM proved to be particularly suitable (cf. arrow and frame).

Figure is illustrates determining the increase in fluorescence as a function of the NAD concentration in the method according to the invention for identifying fungicides. The assay volume amounted to 35 μl. 8.3 mM of ribose-5-phosphate were employed.

FIG. 7 illustrates carrying out the method according to the invention for identifying fungicides with different amounts of RPI, starting with 25 ng of RPI/well (positive control). The negative control is the reaction mixture without addition of RPI.

FIG. 8 illustrates the temperature dependency of the RPI reaction. The reaction and the control without RPI were each incubated for 3 hours at room temperature (RT) and at 37° C.

FIG. 9 illustrates determining the z factor of the method according to the invention based on fluorescence measurements in 24 test batches. The z factor is a parameter for determining the quality of a screening method or an inhibition test. The z factor takes into consideration not only the difference between signal and background, but also the variance of all data.

FIGS. 10A, 10B and 10C illustrate the alignment of the U. maydis RPI sequence with known ribose-phosphate isomerase sequences from S. pombe, S. cerevisiae, Mus musculus, D. melanogaster, C. elegans, A. thaliana and E. coli. Identical amino acids are shown against a grey background.

FIGS. 11A-11E illustrate the alignment of the U maydis RPI sequence with ribose-phosphate isomerase sequences from other fungi (U.m.=U maydis; SPAC144_{—}12=S. pombe; SC_RKI1=S. cerevisiae; CRYNE_{—}001022=C. neoformans; CANAL_Contig6-2195=C. albicans; embl.CNS06G7H=S. bayanus; NEUCR_contig=N. crassa; embl.CNS06MQK=S. kluyveri; embl.CNS06EST=Z. rouxii; embl.CNS0766C=P. angusta; embl.CNS06ZNB=K marxianus; embl.CNS06ZLP=K. marxianus; embl.KLAJ9603=K. lactis). Some of the sequences shown have hitherto not been annotated, i.e. the fact that these sequences encode an RPI was hitherto unknown. They are the sequences from Cryptococcus neoformans, Candida albicans, Neurospora crassa, Zygosaccharomyces rouxii and also the RPI-encoding sequence from Hypocrea jecorina (BM076780). The sequences from S. pombe, S. bayanus, S. cerevisiae, S. kluyveri, P. angusta, Kluyveromyces marxianus, K. lactis and, for example, Yarrowia lipolytica (embl|CNSO6QCU), all of them non-phytopathogenic fungi, were already known as encoding an RPI. Identical amino acids are shown against a grey background.

FIG. 12 illustrates the detail from a 384-well microtitre plate in which the conversion of D-ribose-5-phosphate into D-ribulose-5-phoshate was monitored by detecting the ribulose as purple colour (546 nm) following a colour reaction with a carbazole/cystein/HCl solution. The lines (top to bottom) show the decreasing amounts of ribose-5-phosphate employed; column 1 shows the reaction in the absence of RPI1, columns 2 and 3 in the presence of 400 ng of RPI1.

FIG. 13 illustrates the detection of the enzyme reaction of RPI by means of a coupled enzyme assay, in this case fluorometrically via the decrease in NADH. The presence of the enzyme NADH is consumed, resulting in a decrease in relative fluorescence (cf Example 2C).

FIG. 14 illustrates a spore analysis of the rpi knock-out strains. The plates marked A show spores on full-medium plates without selection. The plates marked B contain hygromycin, allowing only the growth of spores with resistance owing to the presence of the resistance cassette. The spores growing on the selection plates B were analyzed for diploidy, and any presence of the wild-type gene rpi was checked by PCR.

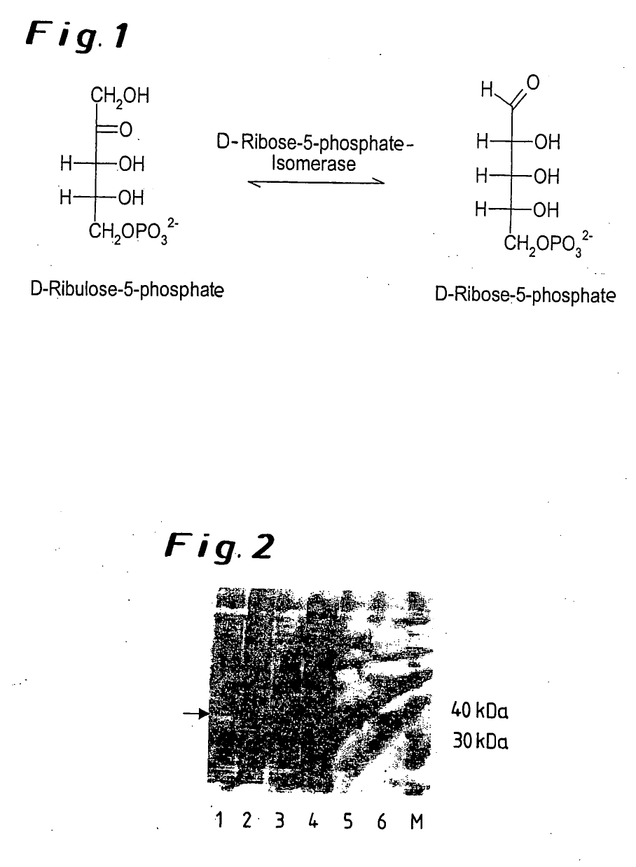

SEQ ID NO. 1 is the Ustilago maydis sequence which encodes RPI.

SEQ ID NO. 2 is the amino acid sequence of the Ustilago maydis RPI, encoded by the sequence as shown in SEQ ID NO. 1.

### DETAILED DESCRIPTION OF THE INVENTION

Definitions

The term “identity” as used in the present context refers to the number of sequence positions that are identical in an alignment. In most cases, it is indicated as a percentage of the alignment length.

The term “similarity” as used in the present context, in contrast, assumes the existence of a similarity metric, that is to say a measure for the desired assumed similarity, for example, between a valine and a threonine or a leucine.

The term “homology” as used in the present context, in turn, indicates evolutionary relationship. Two homologous proteins have developed from a shared precursor sequence. The term is not necessarily about identity or similarity, apart from the fact that homologous sequences usually have a higher degree of similarity (or occupy more identical positions in an alignment) than non-homologous sequences.

The term “RPI” as used in the present context represents ribose-5-phosphate isomerase, which converts D-ribose-5-phosphate into D-ribulose-5-phosphate.

The term “complete RPI” as used in the present context describes the RPI encoded by the complete coding region of a transcription unit, starting with the ATG start codon and comprising all the information-bearing exon regions of the gene encoding RPI which is present in the source organism, as well as the signals required for correct transcriptional termination.

The term “biological activity of an RPI” as used in the present context refers to the ability of a polypeptide to catalyse the above-described reaction, i.e. the conversion of D-ribose-5-phosphate into D-ribulose-5-phosphate.

The term “active fragment” as used in the present context describes nucleic acids encoding RPI which are no longer complete, but still encode polypeptides with the biological activity of an RPI and which are capable of catalysing a reaction characteristic of RPI, as described above. Such fragments are shorter than the above-described complete nucleic acids encoding RPI. In this context, nucleic acids may have been removed both at the 3′ and/or 5′ ends of the sequence, or else parts of the sequence which do not have a decisive adverse effect on the biological activity of the RPI may have been deleted, i.e. removed. A lower or else, if appropriate, an increased activity which still allows the characterization or use of the resulting RPI fragment is considered as sufficient for the purposes of the term as used herein. The term “active fragment” may likewise refer to the amino acid sequence of RPI; in this case, it applies analogously to what has been said above for those polypeptides which no longer contain certain portions in comparison with the above-described complete sequence, but where no decisive adverse effect is exerted on the biological activity of the enzyme.

The term “gene” as used in the present context is the name for a segment from the genome of a cell which is responsible for the synthesis of a polypeptide chain.

The term “cDNA” as used in the present context describes complementary DNA obtained from an mRNA by reverse transcription. It only contains the sequences which correspond to the exons of the genomic DNA. Following cDNA sequencing, the amino acid sequence of the protein encoded by it can be deduced, if desired. After introduction of a cDNA into a cell, large amounts of the respective protein encoded by it can be synthesized.

The term “to hybridize” as used in the present context describes the process in which a single-stranded nucleic acid molecule undergoes base pairing with a complementary strand. For example, starting from the sequence information which is mentioned herein or which can be deduced, DNA fragments can be isolated, in this manner, from phytopathogenic fungi other than Ustilago maydis, which fragments encode RPIs with the same properties as or similar properties to one of the RPIs according to the invention.

Hybridization conditions are calculated approximately by the following formula:

The melting temperature Tₘ:

Tₘ=81.5° C.+16.6{log[c(Na⁺)]}+0.41(% G+C)−(500/n)

(Lottspeich, F., Zorbas H. (ed.). (1998). Bioanalytik. Spektrum Akademischer Verlag, Heidelberg, Berlin).

In this formula, c is the concentration and n the length of the hybridizing sequence segment in base pairs. For a sequence >100 bp, the term 500/n is dropped. The highest stringency involves washing at a temperature of 5-15° C. below Tm and an ionic strength of 15 mM Na⁺(corresponds to 0.1×SSC). If an RNA sample is used for hybridization, the melting point is 10-15° C. higher.

Preferred hybridization conditions are stated hereinbelow:

Hybridization solution: DIG Easy Hyb (Roche, ZZ) hydridization temperature: 42° C. to 70° C., preferably at 42-65° C. (DNA-DNA) or 50° C. (DNA-RNA). Stringent temperatures for the hybridization which are particularly suitable in the present case are between 50 and 65° C., a temperature of 65° C. being an especially suitable stringent temperature.

| | | |
|---|---|---|
| 1. | Wash step 1: | 2 × SSC, 0.1% SDS 2 × 5 min at room temperature; |
| 2. | Wash step 2: | 1 × SSC, 0.1% SDS 2 × 15 min at 50° C.; |
| | | preferably 0.5 × SSC, 0.1% SDS 2 × 15 |
| | | min at 65° C.; particularly preferably |
| | | 0.2 × SSC, 2 × 15 min at 68° C. |

The degree of identity of the nucleic acids or amino acids is preferably determined with the aid of the program NCBI BLASTN Version 2.0.4. (Altschul et al. (1997) “Gapped BLAST and PSI-BLAST: a new generation of protein database search programs”, Nucleic Acids Res. 25:3389).

The term “fungicide” or “fungicidal” as used in the present context refers to chemical compounds which are capable of controlling those fungi which attack and damage plants, plant parts or plant products or reduce their yield or value. The abovementioned plant parts include, for example, leaves, seeds and fruits (such as, for example, berries, fruit, cereal kernels). The abovementioned plant products include plant-derived raw materials or substances such as, for example, timber or fibres. Examples of such fungi are Plasmodiophoromycetes, Oomycetes, Chytridiomycetes, Zygomycetes, Ascomycetes, Basidiomycetes and Deuteromycetes, for example Pythium species, such as, for example, Pythium ultimum, Phytophthora species, such as, for example, Phytophthora infestans, Pseudoperonospora species, such as, for example, Pseudoperonospora humuli or Pseudoperonospora cubensis, Plasmopara species, such as, for example, Plasmopara viticola, Bremia species, such as, for example, Bremia lactucae, Peronospora species, such as, for example, Peronospora pisi or P. brassicae, Erysiphe species, such as, for example, Erysiphe graminis, Sphaerotheca species, such as, for example, Sphaerotheca fuliginea, Podosphaera species, such as, for example, Podosphaera leucotricha, Venturia species, such as, for example, Venturia inaequalis, Pyrenophora species, such as, for example, Pyrenophora teres or P. graminea (conidia form: Drechslera, Syn: Helminthosporium), Cochliobolus species, such as, for example, Cochliobolus sativus (conidia form: Drechslera, Syn: Helminthosporium), Uromyces species, such as, for example, Uromyces appendiculatus, Puccinia species, such as, for example, Puccinia recondita, Sclerotinia species, such as, for example, Sclerotinia sclerotiorum, Tilletia species, such as, for example, Tilletia caries; Ustilago species, such as, for example, Ustilago nudaor Ustilago avenae, Pellicularia species, such as, for example, Pellicularia sasakii, Pyricularia species, such as, for example, Pyricularia oryzae, Fusarium species, such as, for example, Fusarium culmorum, Botrytis species, such as, for example, Botrytis cinerea, Septoria species, such as, for example, Septoria nodorum, Leptosphaeria species, such as, for example, Leptosphaeria nodorum, Cercospora species, such as, for example, Cercospora canescens, Alternaria species, such as, for example, Alternaria brassicae or Pseudocercosporella species, such as, for example, Pseudocercosporella herpotrichoides. Others which are of particular interest are, for example, Magnaporthe grisea, Cochliobulus heterostrophus, Nectria hematococcus and Phytophthora species.

Likewise, the term “fungicide” or “fungicidal” refers to chemical compounds which are suitable for controlling fungi which are pathogens to humans or animals, that is to say also to antimycotics. These include, for example, the following fungi which are pathogenic to humans and which can cause specific symptoms: Dermatophytes such as, for example, Trichophyton spec., Microsporum spec., Epidermophyton floccosum or Keratomyces ajelloi, which cause, for example, athlete's foot (tinea pedis), yeasts such as, for example, Candida albicans, which causes soor oesophagitis and dermatitis, Candida glabrata, Candida krusei or Cryptococcus neoformans, which may cause, for example, pulmonal cryptococcosis or else torulosis, moulds such as, for example, Aspergillus fumigatus, A. flavus, A. niger, which cause, for example, bronchopulmonary Aspergillosis or fungal sepsis, Mucor spec., Absidia spec., or Rhizopus spec., which cause, for example, Zygomycoses (intravasal mycoses), Rhinosporidium seeberi, which causes, for example, chronic granulomatous pharyngitis and tracheitis, Madurella myzetomatis, which causes, for example, subcutaneous mycetomes, Histoplasma capsulatum, which causes, for example, reticulo-endothelial cytomycosis and Darling's disease, Coccidioides immitis, which causes, for example, pulmonary coccidioidomycosis and sepsis, Paracoccidioides brasiliensis, which causes, for example, South American blastomycosis, Blastomyces dermatitidis, which causes, for example, Gilchrist's disease and North American blastomycosis, Loboa loboi, which causes, for example, keloid blastomycosis and Lobo's disease, and Sporothrix schenckii, which causes, for example, sporotrichosis (granulomatous dermal mycosis).

The term “heterologous promoter” as used in the present context refers to a promoter which has properties other than the promoter which controls the expression of the gene in question in the original organism.

The term “competitor” as used in the present context refers to the property of the compounds to compete with other, possibly yet to be identified, compounds for binding to the RPI and to displace the latter, or to be displaced by the latter, from the enzyme.

The term “agonist” as used in the present context refers to a molecule which accelerates or increases RPI activity.

The term “antagonist” as used in the present context refers to a molecule which slows down or prevents RPI activity.

The term “modulator” as used in the present context is the generic term for agonist or antagonist. Modulators can be small organochemical molecules, peptides or antibodies which bind to the polypeptides according to the invention or influence their activity. Moreover, modulators can be small organochemical molecules, peptides or antibodies which bind to a molecule which, in turn, binds to the polypeptides according to the invention, thus influencing their biological activity. Modulators can be natural substrates and ligands, or structural or functional mimetics of these. However, the term “modulator” as used in the present context takes the form of those molecules which do not constitute the natural substrates or ligands.

The term “candidate compound” as used in the present context refers to a chemical compound which is employed as a potential modulator or inhibitor in a method for identifying RPI modulators or inhibitors and which is assayed for its ability to modulate or inhibit the polypeptide. The candidate compound which shows a suitable activity is then used in further tests as RPI inhibitor or modulator, or RPI agonist or antagonist, and used as fungicide, if appropriate following a further testing of its ability to damage or destroy fungi.

Discussion

Within the scope of the present invention, the nucleic acid sequence encoding the RPI from the phytopathogenic fungus U. maydis and the polypeptide encoded by the former are made available. Also provided is a method which is suitable for determining the RPI activity and for identifying inhibitors of the enzyme, including in HTS and UHTS methods, in which the identified compounds can be used as fungicides. It is furthermore demonstrated within the scope of the present invention that RPI inhibitors, in particular fungal RPI inhibitors, preferably inhibitors from phytopathogenic fungi, can be used as plant protection agents.

The smut fungus Ustilago maydis, a Basidiomycete, attacks maize plants. The disease occurs in all areas where maize is grown, but gains importance only during dry years. Typical symptoms are the gall-like, fist-sized swellings (blisters) which are formed on all aerial plant parts. The galls are first covered by a whitish-grey coarse membrane. When the membrane ruptures, a black mass of ustilospores, which is first greasy and later powdery, is released. Further species of the genus Ustilago are, for example, U. nuda (causes loose smut of barley and wheat), U. nigra (causes black smut of barley), U. hordei (causes covered smut of barley) and U. avenae (causes loose smut of oats).

A potential rpi gene has now been annotated in Ustilago maydis (Um38_{—}8). By way of validation, a knock-out of Um38_{—}8 (rpi1) was carried out in Ustilago maydis. The knock-out of Um38_{—}8 proved to be lethal, the gene product RPI1 of the gene Um38_{—}8 (rpi1) is thus an essential enzyme encoding in the phytopathogenic fungus U. maydis.

The Ustilago maydis rpi1 gene is 1020 bp in size and contains no intron. UM 38_{—}8 (rpi1) encodes a polypeptide of 339 amino acids in length with a molecular weight of approx. 37000 dalton. To express the rpi1 gene heterologously, the gene was amplified by means of PCR, using gene-specific oligonucleotides, and cloned into the expression vector pET21b so that the RPI1 protein is expressed starting from plasmid pRPI2 with a C-terminal His₆ tag. It was furthermore demonstrated that the protein expressed heterologously in E.coli has the enzymatic activity of an RPI.

It has furthermore been found within the scope of the present invention that RPI can be used for identifying substances in suitable test methods which affect the activity of the enzyme, which is not necessarily the case in various targets which are theoretically of interest. In addition to an RPI from a phytopathogenic fungus which is characterized by its amino acid sequence and the nucleic acid sequence encoding it, suitable test methods for identifying modulators of the enzyme and which are also suitable for use in HTS methods are thus provided.

It has furthermore been found within the scope of the present invention that RPI is indeed inhibited in vitro by active compounds and that a fungal organism treated with these active compounds can be damaged or killed by the treatment with these active compounds. RPI inhibitors from phytopathogenic fungi can thus be used as fungicides in crop protection. For example, it is shown in the present invention that the inhibition of RPI with substances identified in an abovementioned test method leads to destruction of the treated fungi both in synthetic media and on the plant.

As has already been explained above, it was previously unknown that RPI, in phytopathogenic fungi, can be a target protein of fungicidally active substances, despite the intensive research into RPI. Thus, it is demonstrated for the first time in the present invention that RPI constitutes an enzyme which is important in particular for phytopathogenic fungi, and which is therefore particularly suitable for being used as target protein for the search for further, improved fungicidally active compounds.

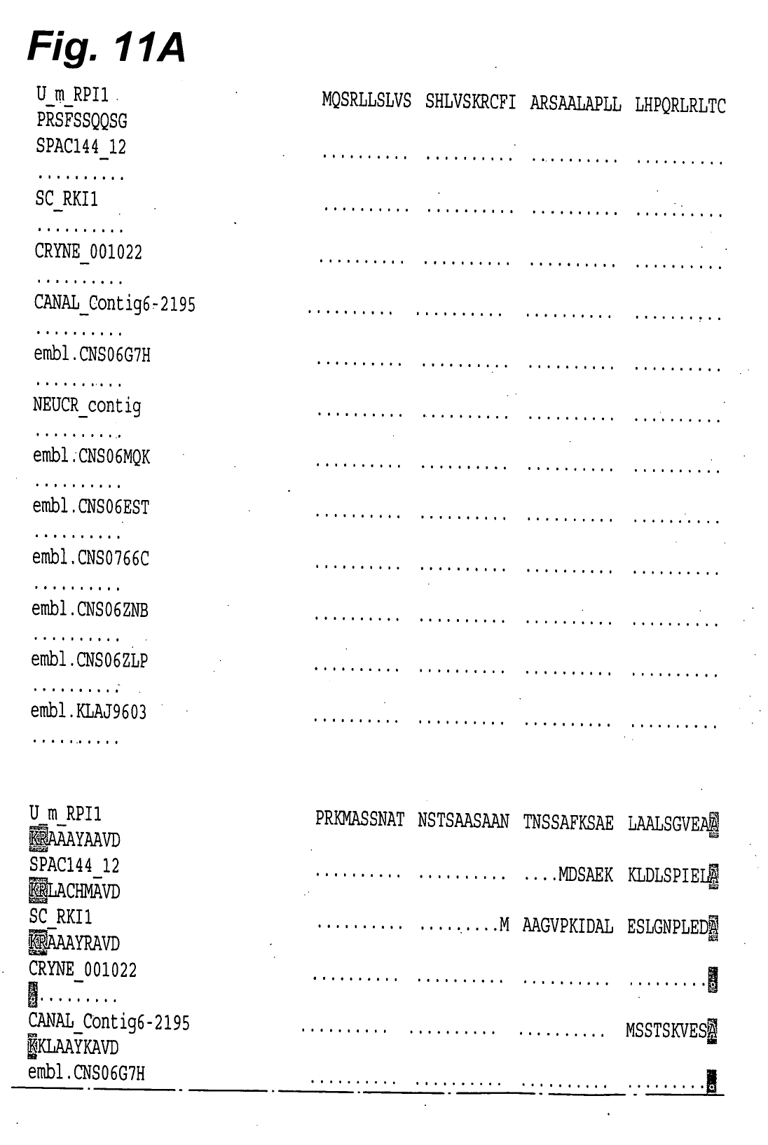

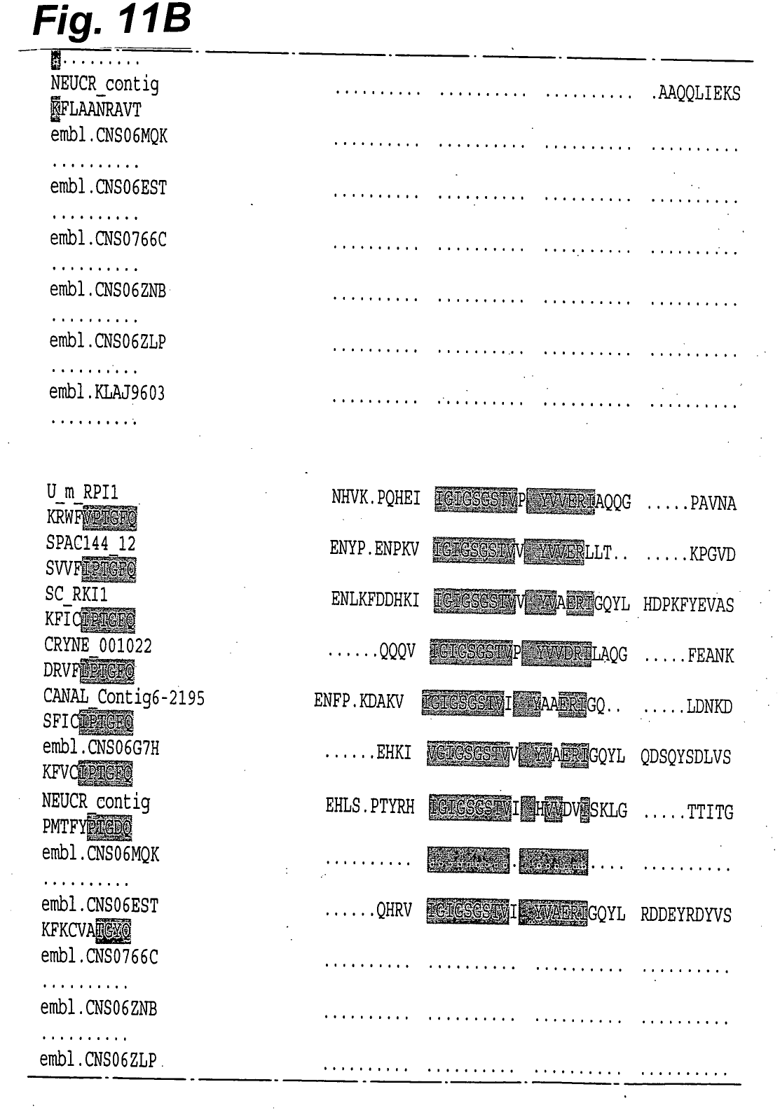

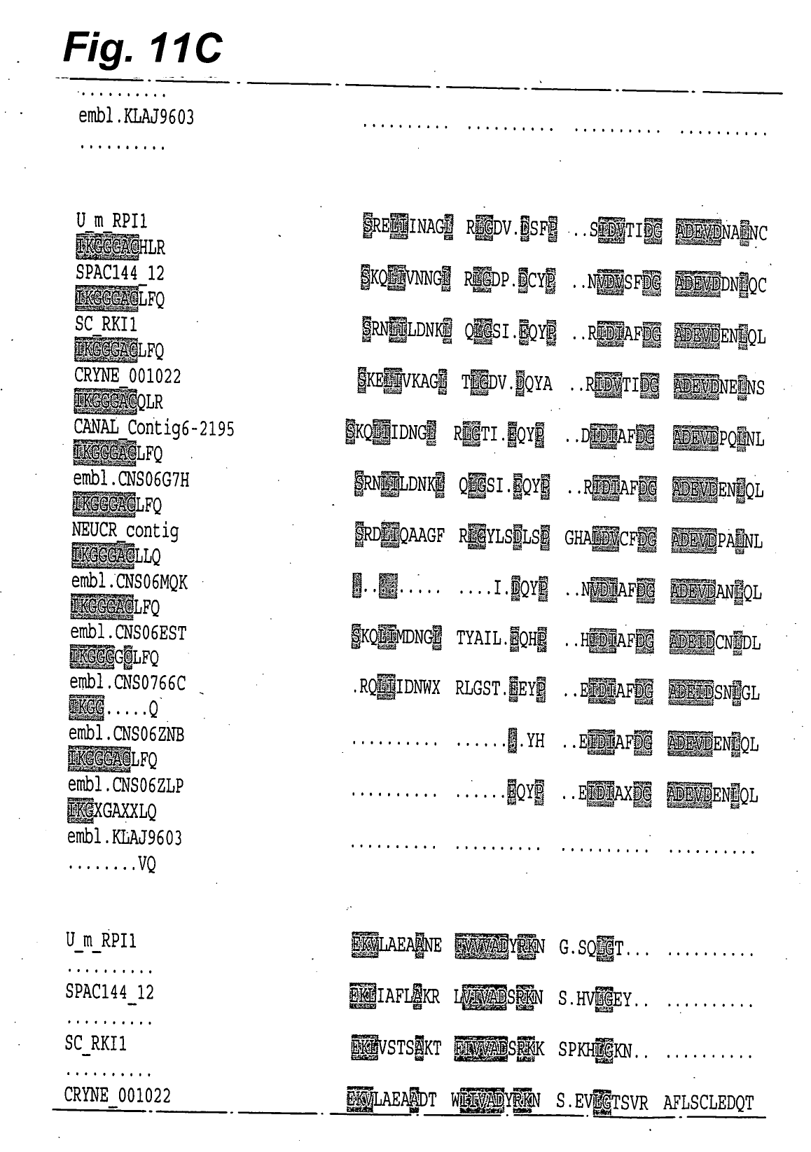

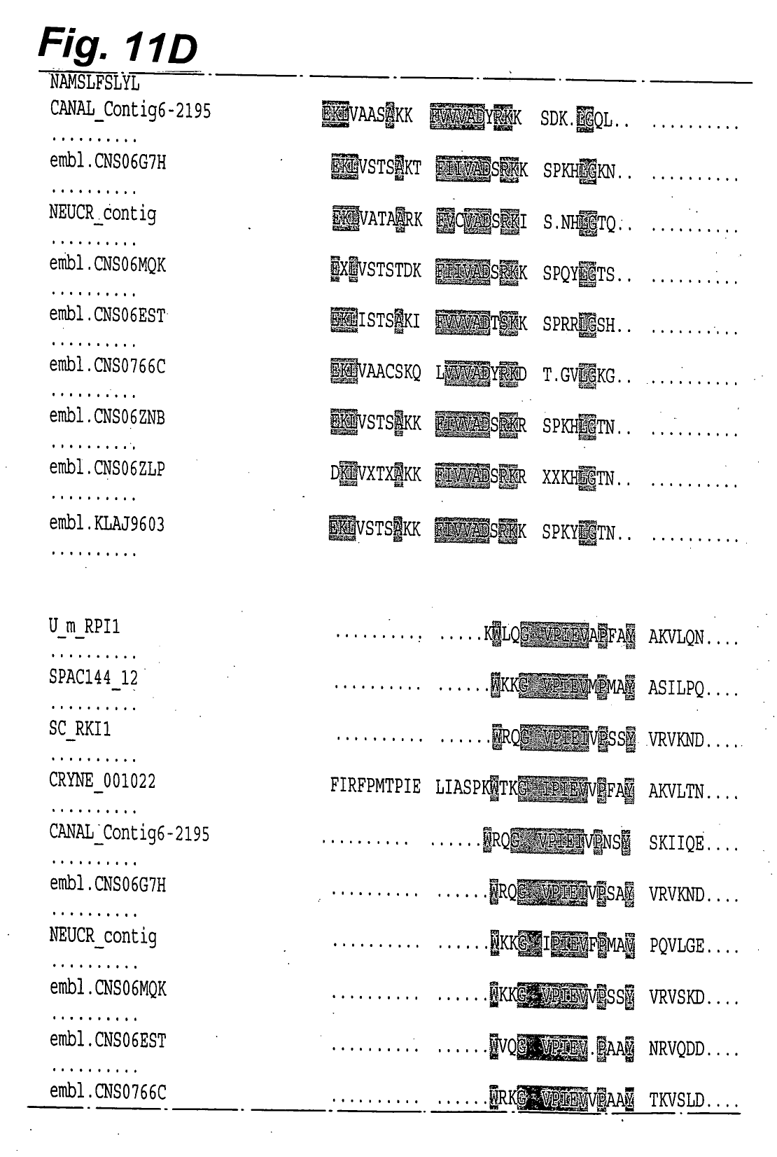

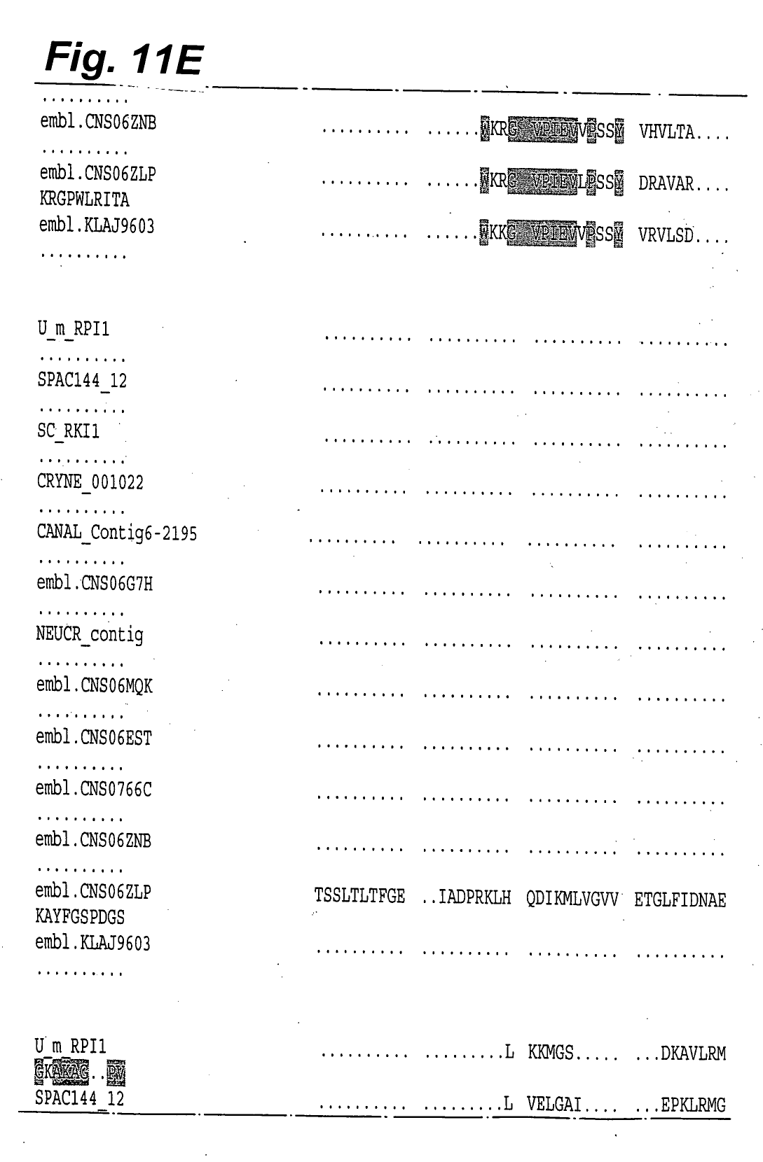

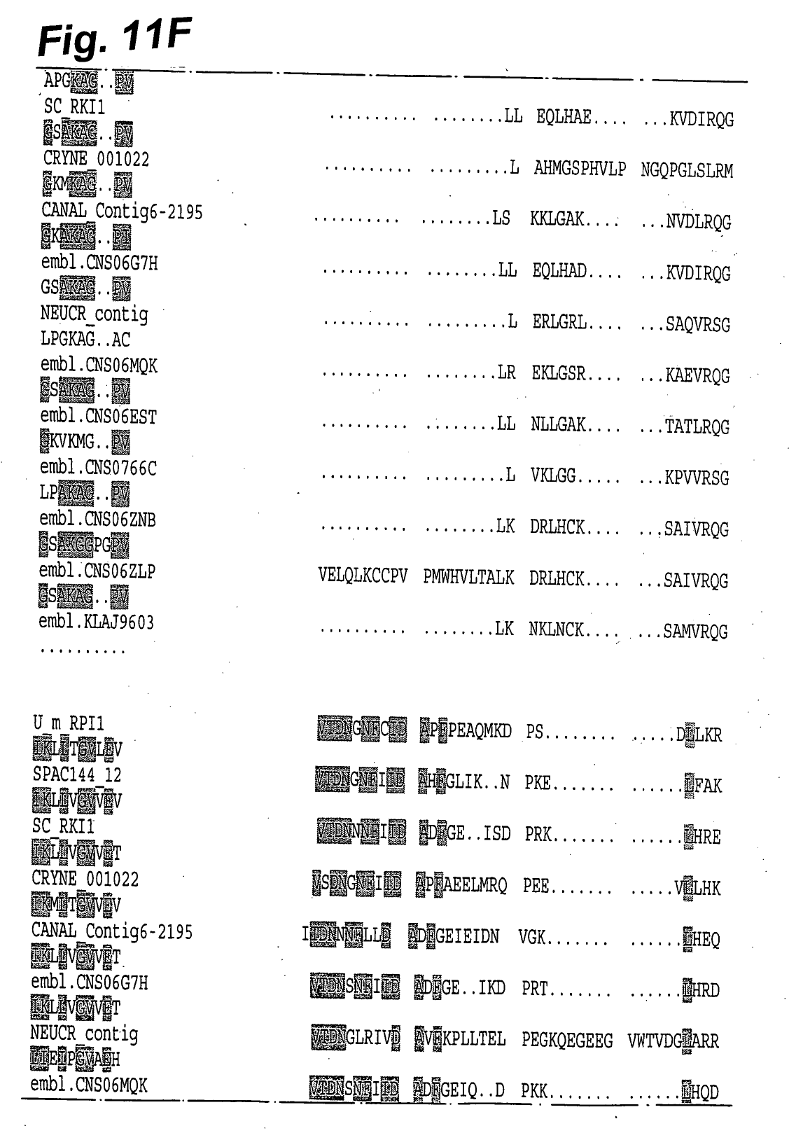

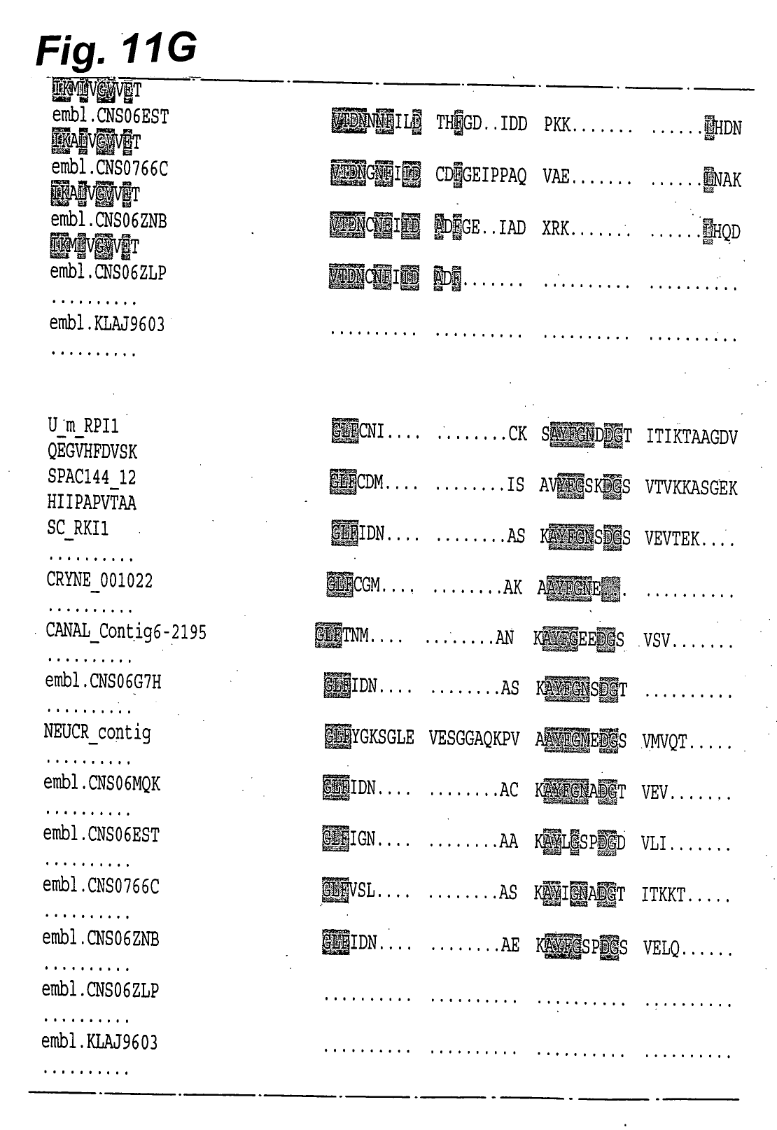

RPIs are divided into several homologous regions (cf. FIGS. 11A-11E). With the aid of a sequence alignment, it was possible, within the scope of the present invention, to identify consensus regions which may serve for identifying and characterizing RPIs in other fungi too.

The specific consensus sequences which can be exploited for identifying or assigning further polypeptides according to the invention are

(a) -(I/V)GIGSGSTV-,

(b) -(P)TG(F/D)QSX₂LI-,

(c) -(I/V)D(I/V)X₂DGADE(I/V)DX₂LX₂IKGG-,

(d) -EK(V/L)X₄AX₂F(I/V)XVADX(R/S)K-,

(e) -WX₂G(I/V)PIEVXP-,

(f) -AKAGP(I/V)VTDNXNFX(I/V/L)D-,

(g) -IKXLXGVXEXGLF-, and

(h) -AYFGNXDG-,

in particular the sequences mentioned under (b), (d), (g) and (h), it being possible for the amino acids given in brackets to be present as an alternative and the letter X describing a position at which any amino acid is accepted. The number of possible amino acids X is indicated by a subscript number.

The abovementioned consensus sequences are typical of polypeptides with the biological activity of an RPI.

The present invention therefore also relates to polypeptides from phytopathogenic fungi with the biological activity of an RPI which encompass one, preferably more, and in particular all, of the abovementioned consensus sequences. The present invention furthermore relates to methods of identifying fungicides which are based on inhibition tests carried out with a polypeptide with the biological activity of an RPI which encompasses one, more or all of the abovementioned consensus sequences.

Owing to the above results and the homologies which exist in species-specific nucleic acids encoding RPI, it is also possible to obtain, and use in a method according to the invention, RPIs from another organism, in particular also from other phytopathogenic fungi, in order to achieve the above aim, i.e. they can likewise be used for identifying fungicides. However, it is also feasible to use another fungus which is not phytopathogenic, or its RPI, or the sequence encoding it, in order to identify fungicidally active RPI inhibitors, such as, for example, S. cerevisiae. Owing to the RPI-encoding nucleic acid sequence which is known from S. cerevisiae, or owing to the sequence of SEQ ID NO: 1, which is given in the present application, it is possible to deduce oligonucleotide primers and thus to obtain further RPI-encoding nucleic acids from other phytopathogenic fungi, for example by means of PCR. Such nucleic acids and their use in methods according to the invention are considered as being encompassed by the present invention.

The present invention therefore relates to nucleic acids which encode complete polypeptides from phytopathogenic fungi with the biological activity of an RPI. These nucleic acids preferably have an identity of at least 70%, at least 75%, at least 80%, at least 85%, at least 90%, at least 95% and in particular at least 98% over a length of 60, 300, 600 or 1200 base pairs and preferably over the entire length of the coding sequence and in particular over the sequence of SEQ ID NO: 1 according to the invention.

The present invention particularly relates to nucleic acids which encode an RPI from phytopathogenic Basidiomycetes, preferably from the genus Ustilago.

The present invention very particularly preferably relates to nucleic acids which encode the Ustilago maydis RPI.

The present invention especially preferably relates to nucleic acids from Ustilago maydis which encode a polypeptide as shown in SEQ ID NO: 2 or active fragments thereof. The present invention relates especially to the nucleic acids of SEQ ID NO: 1.

The nucleic acids according to the invention especially take the form of single-stranded or double-stranded deoxyribonucleic acids (DNA) or ribonucleic acids (RNA). Preferred embodiments are fragments of genomic DNA, which may contain introns, and cDNAs.

The nucleic acids according to the invention preferably take the form of DNA fragments which correspond to the cDNA of the nucleic acids according to the invention.

The nucleic acids according to the invention particularly preferably encompass a sequence from phytopathogenic fungi encoding a polypeptide with the biological activity of an RPI

(a) the sequence of SEQ ID NO: 1,

(b) sequences which encode a polypeptide encompassing an amino acid sequence of SEQ ID NO: 2,

(c) sequences which encode a polypeptide encompassing at least one of the consensus sequences (a) to (h),

(d) part-sequences of the sequences defined under a) to c) which are at least 30 base pairs in length,

(e) sequences which hybridize with the sequences defined under a) to c) at a hybridization temperature of 42-65° C.,

(f) sequences with at least 70%, preferably 80%, particularly preferably 90% and very particularly preferably 95% identity with the sequences defined under a) to c),

(g) sequences which are complementary to the sequences defined under a) to e), and

(h) sequences which, owing to the degeneracy of the genetic code, encode the same amino acid sequence as the sequences defined under a) to c).

The nucleic acids according to the invention can be generated in the customary manner. For example, the nucleic acid molecules can be generated exclusively by chemical synthesis. However, it is also possible to synthesize chemically short sections of the nucleic acids of the invention, and such oligonucleotides can be radiolabelled or labelled with a fluorescent dye. These labelled oligonucleotides can also be used for screening cDNA libraries generated starting from mRNA, for example, from phytopathogenic fungi. Clones with which the labelled oligonucleotides hybridize are selected for isolating the DNA fragments in question. After characterization of the DNA which has been isolated, the nucleic acids according to the invention are obtained in a simple manner.

Alternatively, the nucleic acids according to the invention can also be generated by means of PCR methods using chemically synthesized oligonucleotides.

The term “oligonucleotide(s)” as used in the present context refers to DNA molecules composed of 10 to 50 nucleotides, preferably 15 to 30 nucleotides. They are synthesized chemically and can be used as probes for hybridization experiments or as primers for PCR (polymerase chain reaction).

To prepare the polypeptides according to the invention, in particular the polypeptide encoded by the nucleic acid sequence of SEQ ID NO: 1, it is furthermore possible to culture host cells containing at least one of the nucleic acids according to the invention under suitable conditions. Thereafter, the desired polypeptides can be isolated in the customary manner from the cells or the culture medium. The polypeptides can also be prepared in in vitro systems.

To prepare the Ustilago maydis RPI according to the invention, it is possible, for example, to express the gene recombinantly in Escherichia coli and to prepare an enzyme preparation from E. coli cells (cf Example 1).

As has already been mentioned above for the nucleic acids, the present invention is not only restricted to the sequence of SEQ ID NO: 2. The results which are shown here for the first time within the scope of the present invention likewise apply to other polypeptides from phytopathogenic fungi with the biological activity of an RPI. Thus, it is also possible to obtain, and use in methods according to the invention, a homologous RPI from other fungal species as mentioned above in the definition of the term “fungicide”. Polypeptides from phytopathogenic fungi with the biological activity of an RPI are therefore considered as being encompassed by the subject-matter of the present invention.

Such polypeptides, which are homologous to the Ustilago maydis RPI, in particular to the polypeptide of SEQ ID NO: 2, and which can be used for identifying fungicidal active substances, need not constitute a complete fungal RPI, but may also only constitute fragments of these as long as they at least retain the biological activity of the complete RPI. Polypeptides which exert the same type of biological activity as an RPI with an amino acid sequence as shown in SEQ ID NO: 2 are still considered as being according to the invention. Polypeptides which are considered as according to the invention are, in particular, those polypeptides which correspond to an RPI for example of the phytopathogenic fungi mentioned above under definitions referring to the term “fungicide”, or to fragments of these, and which still retain their biological activity.

The polypeptides according to the invention thus preferably encompass an amino acid sequence from phytopathogenic fungi selected from:

(a) the sequence of SEQ ID NO: 2,

(b) sequences encompassing at least one of the consensus sequences (a) to (h),

(c) part-sequences of the sequences defined under (a) and (b) which are at least 15 amino acids in length,

(d) sequences which have at least 60%, preferably 70%, particularly preferably at least 80% and very particularly preferably 90% identity with the sequence defined under (a), and

(e) sequences which have the same biological activity as the sequence defined under (a).

The term “polypeptides” as used in the present context refers not only to short amino acid chains which are generally referred to as peptides, oligopeptides or oligomers, but also to longer amino acid chains which are normally referred to as proteins. It encompasses amino acid chains which can be modified either by natural processes, such as post-translational processing, or by chemical prior-art methods. Such modifications may occur at various sites and repeatedly in a polypeptide, such as, for example, on the peptide backbone, on the amino acid side chain, on the amino and/or the carboxyl terminus. For example, they encompass acetylations, acylations, ADP ribosylations, amidations, covalent linkages to flavins, haem moieties, nucleotides or nucleotide derivatives, lipids or lipid derivatives or phosphatidylinositol, cyclizations, disulphide bridge formations, demethylations, cystine formations, formylations, gamma-carboxylations, glycosylations, hydroxylations, iodizations, methylations, myristoylations, oxidations, proteolytic processings, phosphorylations, selenoylations and tRNA-mediated amino acid additions.

The polypeptides according to the invention may exist in the form of “mature” proteins or as parts of larger proteins, for example as fusion proteins. They can furthermore exhibit secretion or leader sequences, pro-sequences, sequences which allow simple purification, such as polyhistidine residues, or additional stabilizing amino acids. The proteins according to the invention may also exist in the form in which they are naturally present in their source organism, from which they can be obtained directly, for example.

In comparison with the corresponding regions of naturally occurring RPIs, the polypeptides according to the invention can have deletions or amino acid substitutions, as long as they still exert at least the biological activity of a complete RPI. Conservative substitutions are preferred. Such conservative substitutions encompass variations, one amino acid being replaced by another amino acid from among the following group:

1. Small, aliphatic residues, non-polar residues or residues of little polarity: Ala, Ser, Thr, Pro and Gly;

2. Polar, negatively charged residues and their amides: Asp, Asn, Glu and Gln;

3. Polar, positively charged residues: His, Arg and Lys;

4. Large aliphatic non-polar residues: Met, Leu, lie, Val and Cys; and

5. Aromatic residues: Phe, Tyr and Trp.

Preferred conservative substitutions can be seen from the following list:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

One possible method for the purification of RPI is based on preparative electrophoresis, FPLC, HPLC (for example using gel filtration columns, reversed-phase columns or hydrophobic columns), gel filtration, differential precipitation, ion-exchange chromatography or affinity chromatography.

A rapid method of isolating the polypeptides according to the invention which are synthesized by host cells using a nucleic acid to be used in accordance with the invention starts with expressing a fusion protein, where the fusion partner may be purified in a simple manner by affinity purification. For example, the fusion moiety may be a 6×His tag, in which case the fusion protein can be purified on a nickel-NTA affinity column. The fusion moiety can be removed by partial proteolytic cleavage, for example at linkers between the fusion moiety and the polypeptide according to the invention which is to be purified. The linker can be designed in such a way that it includes target amino acids, such as arginine and lysine residues, which define sites for trypsine cleavage. Standard cloning methods using oligonucleotides may be employed for generating such linkers.

Other purification methods which are possible are based, in turn, on preparative electrophoresis, FPLC, HPLC (e.g. using gel filtration columns, reversed-phase columns or mildly hydrophobic columns), gel filtration, differential precipitation, ion-exchange chromatography and affinity chromatography.

The terms “isolation or purification” as used in the present context mean that the polypeptides according to the invention are separated from other proteins or other macromolecules of the cell or of the tissue. The protein content of a composition containing the polypeptides according to the invention is preferably at least 10 times, particularly preferably at least 100 times, higher than in a host cell preparation.

The polypeptides according to the invention may also be affinity-purified without fusion moieties with the aid of antibodies which bind to the polypeptides.

The present application thus also relates to a method for preparing the polypeptide with a sequence of SEQ ID NO: 2 or polypeptides which are homologous thereto from phytopathogenic fungi with the RPI activity, which comprises

(a) culturing a host cell containing at least one expressible nucleic acid sequence encoding a polypeptide from phytopathogenic fungi with the biological activity of an RPI under conditions which ensure the expression of this nucleic acid, or

(b) expressing an expressible nucleic acid sequence encoding a polypeptide from phytopathogenic fungi with the biological activity of an RPI in an in vitro system, and

(c) recovering the polypeptide from the cell, the culture medium or the in vitro system.

In particular, the present invention relates to a method for preparing the polypeptide with a sequence of SEQ ID NO: 2 or polypeptides which are homologous thereto with the RPI activity from phytopathogenic fungi, wherein

(a) a nucleic acid sequence comprising all of the sequence segments required for expression, and encoding a polypeptide from phytopathogenic fungi with the biological activity of an RPI, is transformed into suitable host cells,

(b) the transformed cells are incubated at a suitable temperature,

(c) the cells are induced after a period which is suitable for sufficient growth of the cells,

(d) the cells are harvested after a period suitable for sufficient expression of the nucleic acid stated under (a), and, if appropriate,

(e) the polypeptide is isolated from the harvested cells and purified.

The cells thus obtained which contain the polypeptide according to the invention, or the purified polypeptide thus obtained, are suitable for use in methods for identifying RPI modulators or inhibitors, or for identifying fungicides.

The present invention thus also relates to the use of polypeptides which exert at least one biological activity of an RPI, in methods for identifying fungicides. In particular, the present invention relates to the use of a fungal RPI, preferably from phytopathogenic fungi, and in particular of an amino acid sequence selected from:

(a) the sequence of SEQ ID NO: 2,

(b) sequences encompassing at least one of the abovementioned consensus sequences (a) to (h),

(c) sequences which have at least 60%, preferably 70%, particularly preferably at least 80% and very particularly preferably 90% identity with the sequence defined under (a), and

(d) sequences which have the same biological activity as the sequences defined under (a) to (c)

in methods for identifying fungicides.

Particular preference is given to the use of polypeptides from phytopathogenic Basidiomycetes, especially from the genus Ustilago, in particular from Ustilago maydis, the polypeptide of SEQ ID NO: 2 being particularly prefeerred, in methods for identifying inhibitors of a polypeptide with RPI activity, it being possible to use the RPI inhibitors as fungicides.

Fungicidal active substances which are found with the aid of an RPI from a variety of organisms, preferably with an RPI according to the invention, can thus also interact with an RPI from other phytopathogenic fungal species, or fungal species which are pathogenic to humans and animals, but the interaction with the different RPIs which are present in these fungi need not always be equally pronounced. This explains inter alia the selectivity of active substances which has been observed.

As already illustrated above, the use of the nucleic acids or polypeptides according to the invention in a suitable method makes it possible to find compounds which bind to the polypeptides according to the invention and/or which inhibit the compound. They can then be used in plants as fungicides and in humans and animals as antimycotics.

The present invention therefore also relates to a method which is suitable for identifying fungicidal active compounds which bind to the polypeptides according to the invention and/or modulate, i.e. activate or inhibit, their biological activity.

In particular, the present invention relates to a method for identifying fungicides by assaying potential inhibitors or modulators of the enzymatic RPI activity (candidate compounds) in a D-ribulose-5-phosphate isomerase (RPI) inhibition test.

Many assay systems aiming at testing compounds and natural extracts are preferably designed for high throughput figures in order to maximize, within a given period, the number of substances tested. Assay systems which are based on cell-free procedures require purified or semipurified protein. They are suitable for a “first” testing, which aims mainly at detecting any effect which a substance may have on the target protein. Once such a first testing has been carried out, and one or more compounds, extracts and the like have been found, the activity of such compounds can be studied in the laboratory in a more targeted fashion. Thus, in a first step, the inhibition or activation of the polypeptide according to the invention in vitro can be tested again in order to subsequently test the activity of the compound on the target organism, in the present case one or more phytopathogenic fungi. Then, if appropriate, the compound can be used as a starting point for the further search for, and development of, fungicidal compounds which are based on the original structure, but which are optimized for example with regard to activity, toxicity or selectivity.

Methods which are suitable for identifying fungicides, or activators or inhibitors, or agonists or antagonists, of the polypeptides according to the invention are generally based on the determination of the activity or the biological functionality of the polypeptide. Suitable for this purpose are, in principle, methods based on intact cells (in vivo methods), but also methods which are based on the use of the polypeptide isolated from the cells, which may be present in purified or partially purified form or else as a crude extract. These cell-free in vitro methods, like in vivo methods, can be used on a laboratory scale, but preferably also in HTS or UHTS methods.

To find fungicides or modulators, for example a synthetic reaction mix (for example in vitro transcription products) or a cellular component such as a membrane, a compartment or any other preparation containing the polypeptides according to the invention can be incubated together with an optionally labelled substrate or ligand of the polypeptides in the presence and absence of a candidate molecule which can be an agonist or antagonist. The ability of the candidate molecule to increase or to inhibit the activity of the polypeptides according to the invention can be identified for example on the basis of increased or reduced binding of the optionally labelled ligand or increased or reduced conversion of the optionally labelled substrate. Molecules which lead to an increased activity of the polypeptides according to the invention are agonists. Molecules which inhibit the biological activity of the polypeptides according to the invention are antagonists.

Detection of the biological activity of the polypeptides according to the invention can be improved by what is known as a reporter system. In this aspect, reporter systems comprise, but are not restricted to, calorimetrically or fluorimetrically detectable substrates which are converted into a product, or a reporter gene which responds to changes in the activity or the expression of the polypeptides according to the invention, or other known binding assays.

A further example of a method by which modulators of the polypeptides according to the invention can be found is a displacement assay in which the polypeptides according to the invention and a potential modulator are combined, under suitable conditions, with a molecule which is known to bind to the polypeptides according to the invention, such as a natural substrate or ligands or a substrate or ligand mimetic. The polypeptides according to the invention can themselves be labelled, for example fluorimetrically or calorimetrically, so that the number of the polypeptides which are bound to a ligand or which have undergone a conversion can be determined accurately. However, binding can likewise be monitored by means of the optionally labelled substrate, ligand or substrate analogue. The efficacy of an agonist or antagonist can be determined in this manner.

Effects such as cell toxicity are, as a rule, ignored in these in vitro systems. The assay systems check not only inhibitory or suppressive effects of the substances, but also stimulatory effects. The efficacy of a substance can be checked by concentration-dependent assay series. Control mixtures without test substances or without an enzyme can be used for assessing the effects. This is why in such an in vitro assay system the testing of the fungicidal properties of the identified compounds is generally carried out by bringing the compounds into contact with a variety of fungi and assessing whether, and to what extent, the fungus is damaged and destroyed by the compound.

A method of identifying fungicides is based on

a) bringing an RPI or a host cell containing this polypeptide into contact with a chemical compound or a mixture of chemical compounds under conditions which allow the interaction of a chemical compound with the polypeptide,

b) comparing the activity of the RPI in the absence of a chemical compound with the activity of the polypeptide according to the invention in the presence of a chemical compound or a mixture of chemical compounds, and

c) determining the chemical compound which modulates or inhibits the RPI activity.

In this context, the compound which specifically inhibits the activity of the polypeptide according to the invention is particularly preferably determined. The term “activity” as used in the present context refers to the biological activity of RPI.

The enzyme RPI activity, or the increase or decrease in the enzyme RPI activity, or the inhibition of this enzymatic activity, is preferably determined by means of the conversion of the substrate NAD⁺into NADH. Here, the lower, or inhibited, activity of the polypeptide according to the invention is basically monitored with reference to the determination, by fluorescence spectrometry, of the reduced formation of NADH. Owing to the fact that the nicotinamide ring absorbs light, the reduced nicotinamide coenzyme NADH has an absorption maximum at 340 nm. In contrast, the oxidized form NAD⁺does not absorb between 300 and 400 nm. The enzyme reaction according to the invention which, owing to a subsequent coupling of further enzyme reactions, leads to the NAD⁺being reduced and to NADH being formed, can therefore be monitored with reference to the increase in absorption, for example at 340 nm. Moreover, the fluoresence of NADH is more pronounced than that of NAD⁺. Thus, the effect of an inhibitor on the reaction can also be determined thereby, the lesser, or inhibited, RPI activity being determined by a less pronounced increase in fluorescence from NADH, which is formed to a lesser degree.

The method is based on coupling the enzyme activity of RPI, which leads to the formation of D-ribulose-5-phosphate, with further reactions which eventually lead to the conversion of NAD⁺into NADH. The assay system comprises three further auxiliary enzymes, namely ribulose-5-phosphate epimerase (RPE), transketolase (TK) and glycerinaldehyde-3-phosphate dehydrogenase (GAPDH). The following enzyme reactions which are coupled to one another proceed in this method:

| | | | |
|---|---|---|---|
| RPI | | | |
| 1st Step: | D-ribose-5-phosphate | → | D-ribulose-5-phosphate |
| RPE | | | |
| 2nd Step: | D-ribulose-5-phosphate | → | xylulose-5-phosphate |
| TK | | | |
| 3rd Step: | D-ribose-5-phosphate + | → | sedoheptulose-7-phosphate + |
| | xylulose-5-phosphate | | glycerinaldehyde-3- |
| | | | phosphate |
| GAPDH | | | |
| 4th Step: | glycerinaldehyde-3-phosphate + | → | 3-phosphoglycerate + NADH + H⁺ + |
| | NAD⁺ + AsO₄³⁻ | | AsO₄³⁻ |

The equilibrium of the GAPDH reaction is almost completely on the glycerinaldehyde-3-phosphate and NAD⁺side. Only when arsenate instead of the natural substrate phosphate is employed is NADH formed to a noticeable degree, since the resulting arsenate analogue of 1,3-bisphosphoglycerate is unstable and breaks down, thereby shifting the equilibrium to the right.

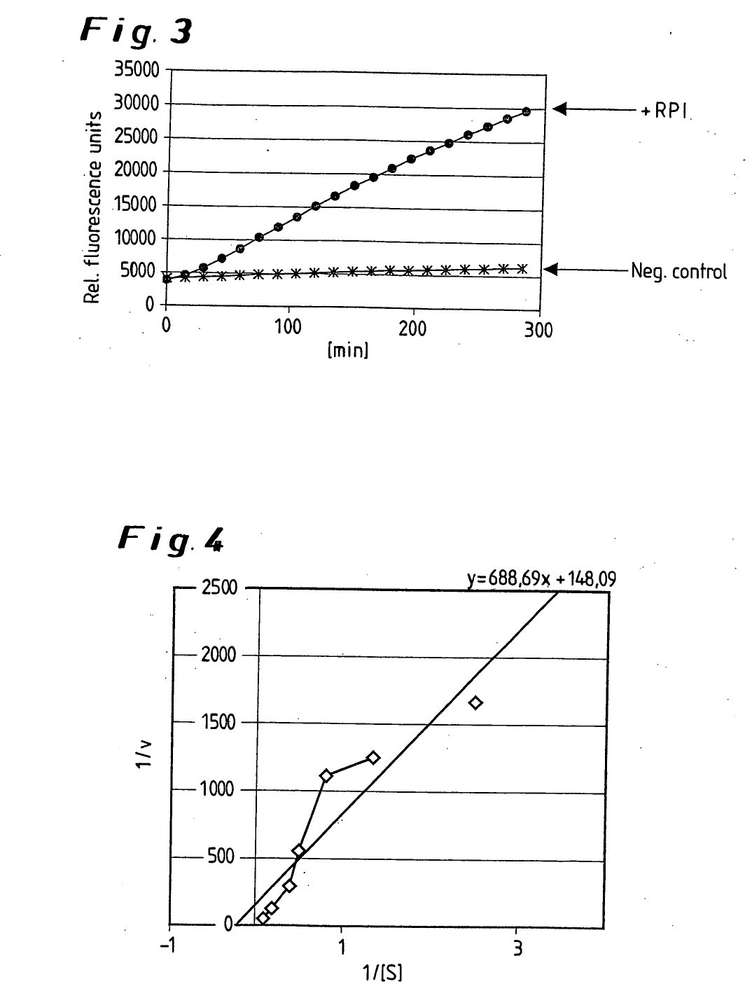

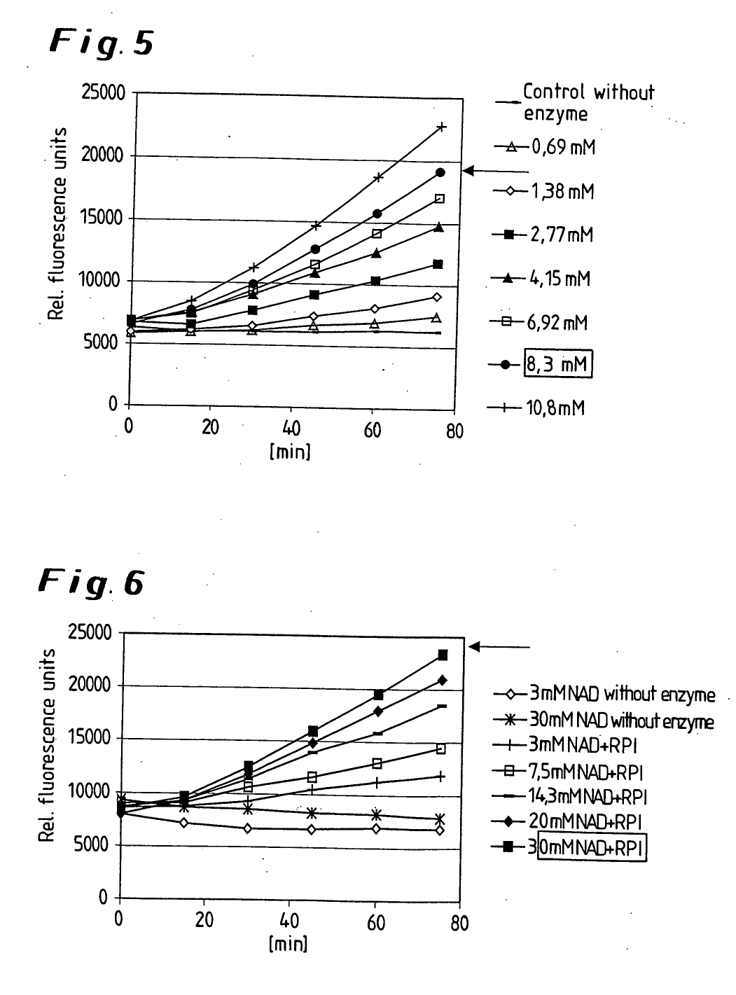

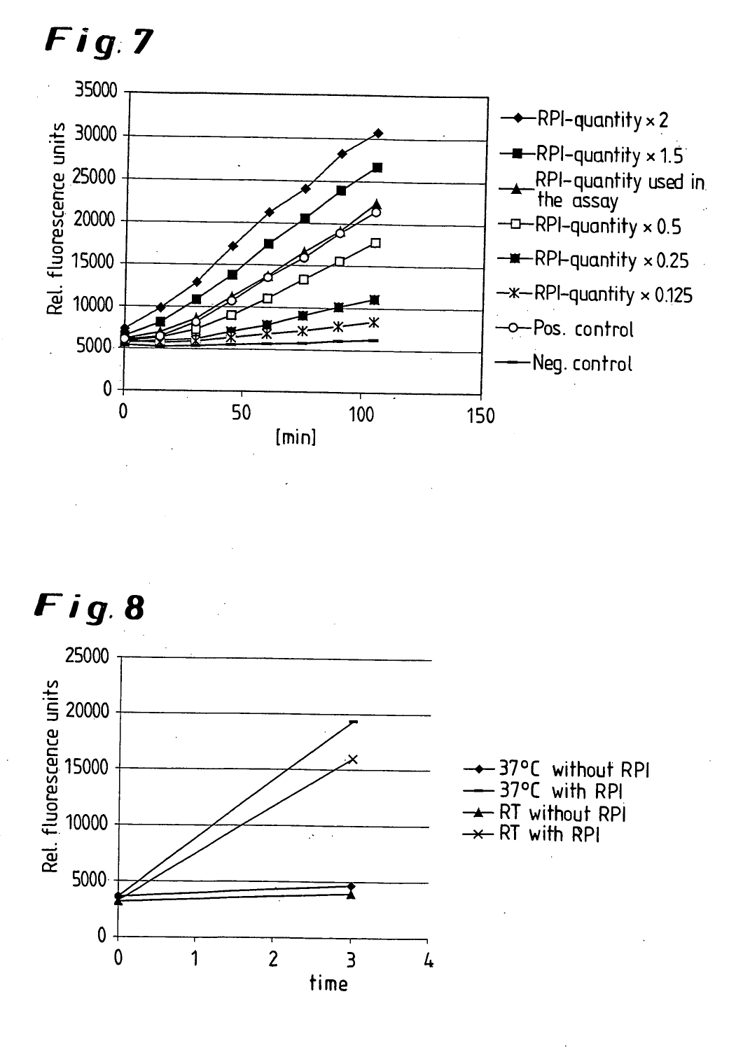

In comparison with RPI, the coupled enzymes are preferably employed in excess in order to avoid the isolation of inhibitors of these coupled enzymes. The formation of NADH in the course of the reaction is then monitored with reference to the fluorescence at an excitation wavelength of 360 nm and an emission wavelength of 465 nm (cf. FIG. 3). The fluorescence yield in the presence of a chemical compound can then be compared with the fluorescence yield in the absence of a chemical compound. The comparison then shows whether the increase in fluorescence is less or, if appropriate, more pronounced in the presence of a chemical compound than in the absence of the chemical compound, i.e. whether said compound has an inhibitory or, possibly, even activatory effect on the tested polypeptide. The period of time within which the increase in fluorescence is measured can be varied. A slow increase in fluorescence can be observed in the course of one to several hours. In order to obtain as high as possible a read-out, that is to say a sufficiently large signal, within a sensible window, all of the parameters such as temperature and concentration of the individual components must be adapted. To this end, the K_{M} value of the substrate D-ribose-5-phosphate is determined, inter alia, a value of 6.3 mM having been determined (cf FIG. 4). The amount of D-ribose-5-phosphate employed can be varied, a concentration of 3 to 10 mM being preferred (cf FIG. 5). The amount of NAD⁺employed can be varied too, a concentration of 7 to 40 mM being especially preferred (cf FIG. 6). The amount of protein can likewise be varied (cf FIG. 7), amounts of more than 25 ng of RPI not being suitable since otherwise inhibitors of the coupled enzymes would increasingly also be found.

Phosphate is described in the literature (Jung et al., Arch. Biochem. Biophys. 373 (2000) 409-417) as a “weak” ribose-phosphate isomerase inhibitor, which is why the assay was carried out in the presence of various phosphate concentrations in order to determine phosphate's effect on the reaction. An additional problem in the coupled assay is that phosphate also acts as substrate for GAPDH and thus accelerates the coupling reaction. Measurements have revealed that only very high phosphate concentrations show an inhibitory effecet. This is why phosphate cannot be used as control inhibitor in this assay.

The temperature can be varied within a relatively wide range, temperatures of from 18 to 37° C. being preferred. The method is especially preferably carried out at 37° C. or at room temperature (cf. FIG. 8).

The measurement can also be carried out in formats conventionally used for HTS or UHTS assays, for example in microtitre plates, into which for example a total volume of 5 to 50 μl is introduced per reaction or per well and the individual components are present in one of the above-stated final concentrations. The compound to be assayed and which potentially inhibits or activates the activity of the enzyme (candidate molecule) is introduced for example in a suitable concentration in DMSO. The solution containing the substrate ribose-5-phosphate is then added. A solution with the polypeptide according to the invention is then added, thus starting the reaction. The mixture is then incubated for example for up to 2 or 3 hours at a suitable temperature, and the increase in fluorescence is measured at an excitation wavelength of 360 nm and an emission wavelength of 460 nm.

A further measurement is carried out in a corresponding mixture, but without addition of a candidate molecule and without addition of RPI (negative control). Another measurement, in turn, is carried out in the absence of a candidate molecule, but in the presence of RPI (positive control). The negative and the positive controls thus provide the reference values for the mixtures in the presence of a candidate molecule.

A further method of identifying fungicides is to carry out an inhibition assay based on a spectrophotometric process. The enzyme activity of RPI is determined with reference to the increase in absorption at 290 nm. The increase in absorption can be attributed to the formation of ribulose-5-phosphate. Inhibition of the enzyme activity of RPI by a chemical compound can be seen from a less pronounced, or no, increase in absorption in comparison with a mixture without candidate compound (cf Example 3 (A)).

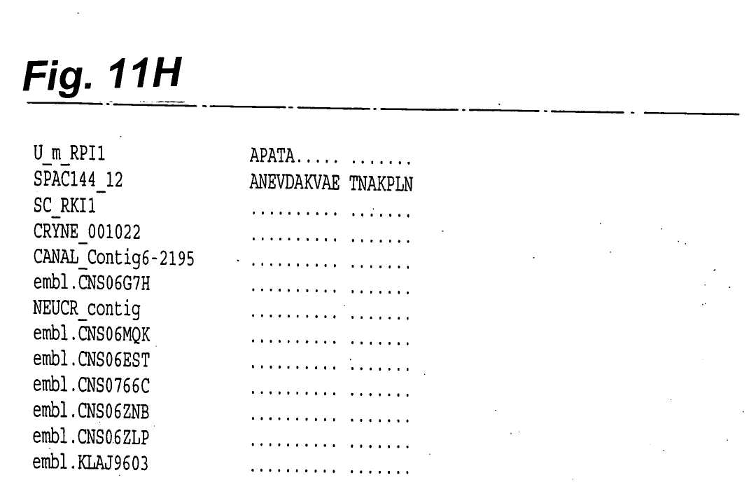

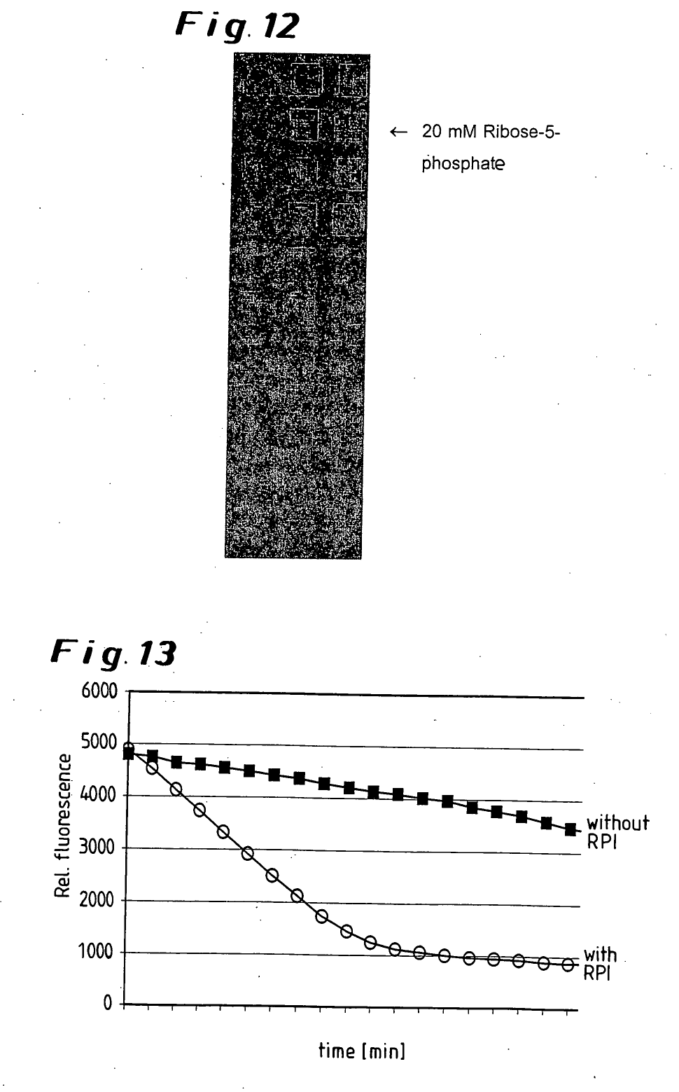

Another method is based on the detection of the keto group of the ribulose, which is formed by the enzyme activity of RPI, using a carbazole/cystein/HCl solution (cf Example 3 (B) and FIG. 12). In this case, inhibition of the enzyme activity of an RPI by a candidate compound can be seen by a less pronounced colouration in comparison with a mixture without candidate compound.

Another coupled enzyme assay based on the coupling of RPI, ribulose-5-phosphate kinase, pyruvate kinase and lactate dehydrogenase can likewise act as a way of identifying fungicides (cf. Ex. 3 (C) and FIG. 13).

An RPI which is preferably used in the methods according to the invention is a fungal RPI, particularly preferably an RPI from phytopathogenic fungi or an RPI from yeast, in particular from S. cerevisae.

The processes which have been described above by way of example have made possible the identification of compounds which inhibit an RPI, in particular the RPIs according to the invention, and which can be used as fungicides.

The present invention therefore particularly relates to a method for identifying fungicides by assaying a candidate compound in an RPI inhibition test.

The method according to the invention is preferably followed by a further step in which the fungicidal action of the compounds identified is tested by bringing fungi into contact with the compound(s) and testing the effect of the compounds on the fungi.

The present invention particularly relates to a method as described above in which the enzyme activity, or the inhibition of this activity by a candidate compound, is assayed by the formation of NADH in a coupled reaction with RPE, TK and GAPDH or with phosphoribulose kinase, pyruvate kinase and lactate dehydrogenase. In the former case, the natural substrate of GAPDH, phosphate, is preferably replaced by arsenate (AsO₄³⁻).

The present invention thus preferably relates to a method for identifying fungicides in which

a) an RPI, preferably a fungal RPI, or a host cell comprising this polypeptide is brought into contact with a chemical compound or with a mixture of chemical compounds under conditions which permit the interaction of a chemical compound with the polypeptide,

b) the RPI activity in the absence of a chemical compound is compared with the activity of the polypeptide according to the invention in the presence of a chemical compound or of a mixture of chemical compounds by coupling the respective enzyme reaction of RPI with further enzyme reactions which lead to the formation of NADH,

c) the chemical compound which leads to a reduced amount of the resulting NADH is identified, and

d) if appropriate, the fungicidal action of the compound is tested by bringing the compound into contact with a fungal organism.

Naturally, the method for identifying fungicides is not limited to the above-described methods, such as the coupled inhibition test, but activity tests or inhibition tests which are suitable for allowing the enzyme activity of an RPI or its inhibition to be determined may also be used. Also included are known methods which are modified in such a way that at least the inhibitory effect of a candidate compound can be recognised in this method, that is to say that the evaluation of the results must be possible. An example of a statistic parameter which can be used is the signal-background ratio. Another suitable parameter for determining the quality of a screening method or an inhibition test is the z factor. The calculation of the z factor also takes into consideration not only the difference between signal and background, but also the variaton of all results. The z factor is calculated as follows: z factor=1−((3 ×standard deviation posCo+3×standard deviation negCo)/(mean posCo−mean negCo)), where “posCo” represents the positive control and “negCo” the negative control (Zhang et al. (1999): A Simple Statistical Parameter for Use in Evaluation and Validation of High Throughput Screening Assays. J. Biomol. Screen. 4(2):67-73).

A z factor greater than 0.7 is considered as excellent, a z factor of 0.15-0.7 as good, a z factor of 0.15 as still sufficient and a z factor of less than 0 as avaluative.

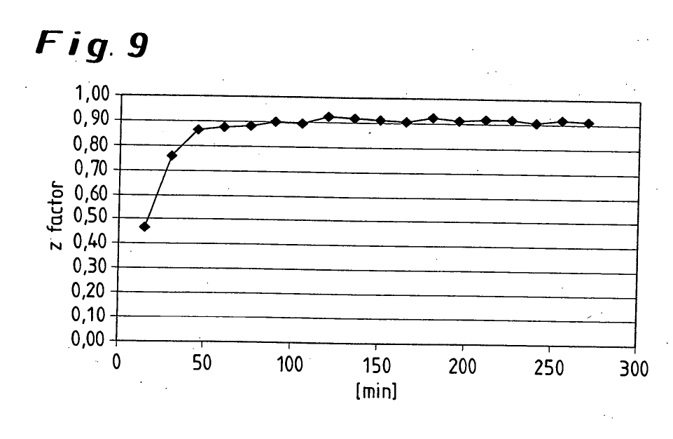

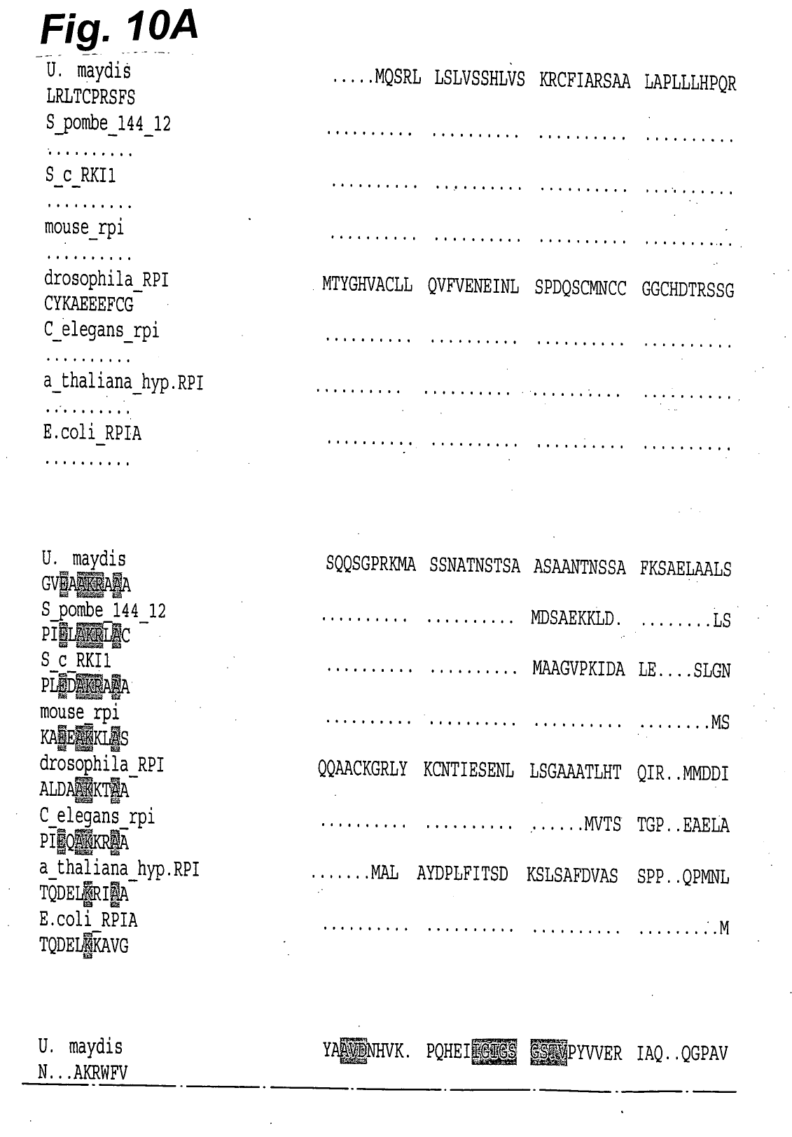

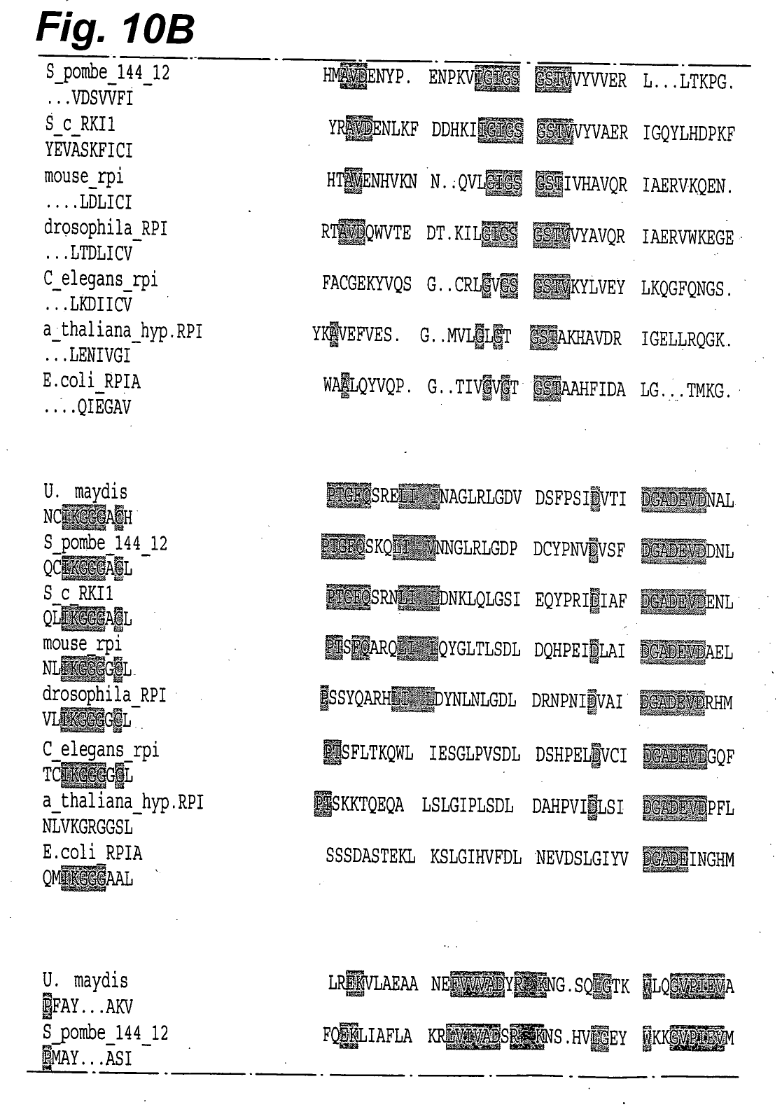

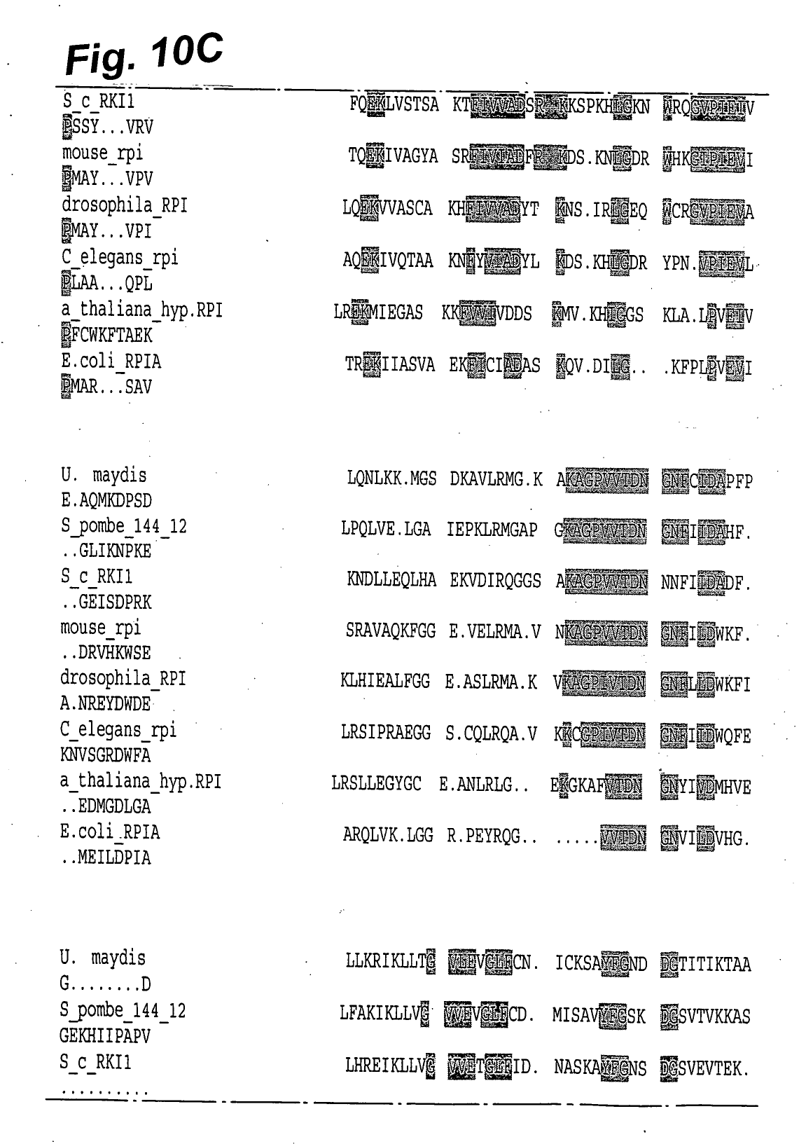

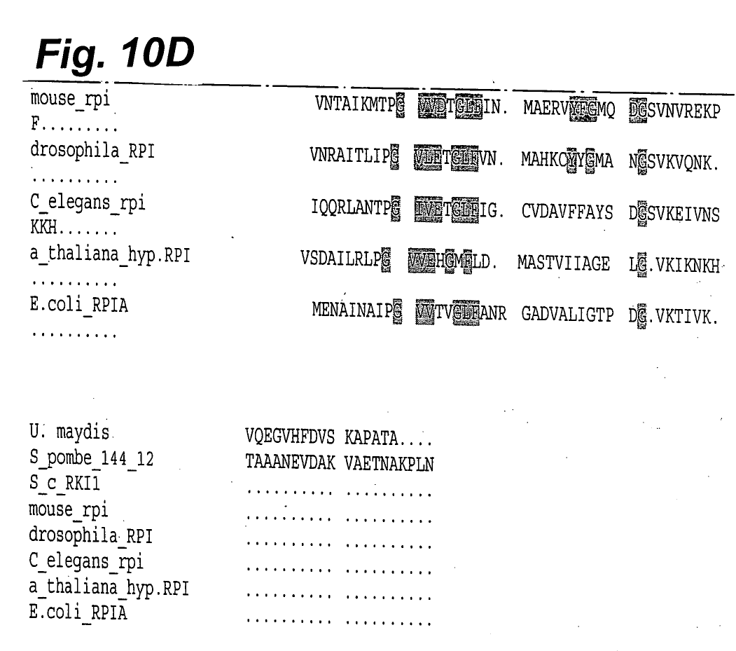

In the method according to the invention, the z factor is above 0.8 after approx. 50 minutes (cf FIG. 9).

The method according to the invention is suitable for determining the activity of an RPI and for detecting the inhibition of this activity by a candidate compound. Candidate compounds which inhibit the activity can then be selected specifically and, if appropriate, developed further.

Table I shows examples of compounds which were identified with a method according to the invention as RPI inhibitors.

TABLE I

| Ex- | | |
|---|---|---|
| ample | | |
| No. | Compound | pI50 |
| 1 | 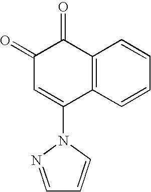 | 4.7 |
| 2 | 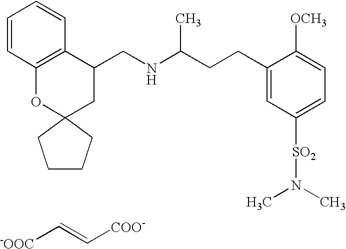 | 4.7 |

The pI50 value which is shown in Table I is the negative decimal logarithm of what is known as the IC50 value, which indicates the molar concentration of a substance which results in 50% inhibition of an enzyme.

For example, a pI50 value of 8 corresponds to half the maximum inhibition of an enzyme at a concentration of 10 nM.

It was furthermore shown within the scope of the present invention that the RPI inhibitors identified with the aid of a method according to the invention are suitable for damaging or destroying fungi.

To this end, a solution of the active substance to be tested was pipetted for example into the cavities of microtitre plates. After the solvent had evaporated, medium was added to each cavity. Previously, a suitable concentration of spores or mycelium of the test fungus had been added to the medium. The resulting active substance concentrations are, for example, 0.1, 1, 10 and 100 ppm.

The plates were subsequently incubated on a shaker at a temperature of 22° C. until sufficient growth was discernible in the untreated control.

The plates were evaluated photometrically at a wavelength of 620 nm. The dose of active substance which leads to a 50% inhibition of fungal growth in comparison with the untreated control (ED₅₀) was calculated from the results of the various concentrations.

Table II shows examples of results of such a test as ED₅₀ values for the compounds found in a method according to the invention (cf Table I).

TABLE II

| Compound | | |
|---|---|---|
| (Example No.) | Organism | ED₅₀ [ppm] |
| 1 | Botrytis cinerea | 9.68 |
| 1 | Pyricularia oryzae | 0.1 |
| 1 | Phytophthora cryptogea | 12.47 |
| 1 | Ustilago avenae | 22.07 |
| 2 | Botrytis cinerea | 9.68 |
| 2 | Pyricularia oryzae | 0.1 |
| 2 | Phythophthora cryptogea | 12.47 |
| 2 | Ustilago avenae | 22.07 |

In the case of Erysiphe graminis, the induction of resistance to this fungus, in a plant, was furthermore tested by way of example. To this end, the plant is inoculated with the pathogen 4 days after treatment of the plant with the test substance. The activity of the substance is given as efficacy in %. Values of between 0 and 100 are calculated, an efficacy of 100 being the maximum activity and 0 being no activity.

In this test, 50% resistance induction in Hordeum vulgaris was found for the compound of Example No. 2 (cf Table I).

The present invention therefore furthermore also relates to RPI inhibitors, in particular to compounds or extracts which are suitable for inhibiting phytopathogenic fungi and which are found with the aid of one of the methods described in the present application for identifying RPI inhibitors.

The present application therefore also relates to fungicides which were found with the aid of a method according to the invention.

Compounds which are identified with the aid of a method according to the invention and which are fungicidally active owing to inhibition of the fungal RPI can then be used for the preparation of fungicidal compositions.

Depending on their respective physical and/or chemical properties, the active substances which have been identified can be converted into the customary formulations, such as solutions, emulsions, suspensions, powders, foams, pastes, granules, aerosols, very fine capsules in polymeric substances and in coating compositions for seed, and ULV cold mist and warm mist formulations.

These formulations are produced in a known manner, for example by mixing the active compounds with extenders, that is, liquid solvents, liquefied gases under pressure, and/or solid carriers, optionally with the use of surface-active agents, that is, emulsifiers and/or dispersants, and/or foam-forming agents. In the case of the use of water as an extender, organic solvents can, for example, also be used as cosolvents. As liquid solvents, the following are suitable in the main: aromatics such as xylene, toluene or alkylnaphthalenes, chlorinated aromatics or chlorinated aliphatic hydrocarbons, usch as chlorobenzenes, chloroethylenes or methylene chloride, aliphatic hydrocarbons, such as cyclohexane or paraffins, for example mineral oil fractions, alcohols, such as butanol or glycol as well as their ethers and esters, ketones such as acetone, methyl ethyl ketone, methyl isobutyl ketone or cyclohexanone, strongly polar solvents, such as dimethyl-formamide and dimethyl sulphoxide, as well as water. By liquefied gaseous extenders or carriers are meant liquids which are gaseous at ambient temperature and under atmospheric pressure, for example aerosol propellants, such as halohydrocarbons as well as butane, propane, nitrogen and carbon dioxide. As solid carriers, the following are suitable: for example ground natural minerals, such as kaolins, clays, talc, chalk, quartz, attapulgite, montmorillonite or diatomaceous earth, and ground synthetic minerals, such as highly disperse silica, alumina and silicates. As solid carriers for granules, the following are suitable: for example crushed and fractionated natural rocks such as calcite, marble, pumice, sepiolite and dolomite, as well as synthetic granules of inorganic and organic meals, and granules of organic material such as sawdust, coconut shells, maize cobs and tobacco stalks. As emulsifiers and/or foam formers, the following are suitable: for example nonionic and anionic emulsifiers, such as polyoxy-ethylene fatty acid esters, polyoxyethylene fatty alcohol ethers, for example alkylaryl polyglycol ethers, alkylsulphonates, alkyl sulphates, arylsulphonates and protein hydrolysates. As dispersants, the following are suitable: for example lignin-sulphite waste liquors and methylcellulose.

Adhesives such as carboxymethylcellulose and natural and synthetic polymers in the form of powders, granules or latices, such as gum arabic, polyvinyl alcohol and polyvinyl acetate, and natural phospholipids such as cephalins and lecithins, and synthetic phospholipids, can be used in the formulations. Further additives can be mineral and vegetable oils.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

The formulations in general contain between 0.1 and 95 per cent by weight of active substance, preferably between 0.5 and 90%.

The active substances according to the invention as such or in their formulations may also be present as a mixture with other known fungicides, bactericides, acaricides, nematicides or insecticides in order to, for example, widen the spectrum of action or prevent the build-up of resistance. In many cases, synergistic effects are obtained, i.e. the efficacy of the mixture exceeds the efficacy of the individual components.

When employing the compounds according to the invention as fungicides, the application rates can be varied within substantial ranges, depending on the application in question.

All plants and plant parts can be treated in accordance with the invention. Plants, in the present context, are understood as meaning all plants and plant populations, such as desired and undesired wild plants or crop plants (including naturally occurring crop plants). Crop plants can be plants which can be obtained by traditional breeding and optimization methods or biotechnological and recombinant methods or combinations of these methods, including the transgenic plants and including the plant varieties which are capable of being protected by Plant Breeders' Rights or not. Plant parts are understood as meaning all aerial and subterranean parts and organs of the plants, such as shoot, leaf, flower and root, examples being leaves, needles, stalks, stems, flowers, fruiting bodies, fruits and seeds, and roots, tubers and rhizomes. The plant parts also include harvested material and vegetative and generative propagation material, for example cuttings, tubers, rhizomes, slips and seeds.

The treatment according to the invention of plants and plant parts with the active substances is effected directly or by acting on their environment, habitat or storage space by the customary treatment methods, for example by dipping, spraying, vapourizing, misting, spreading, brushing on and, in the case of propagation material, in particular seeds, furthermore by coating the material with one or more coats.

The examples which follow are intended to illustrate the various aspects of the present invention and are not to be construed as limiting.

### EXAMPLES

### Example 1

Knock-Out of the Ustilago maydis RPI Gene

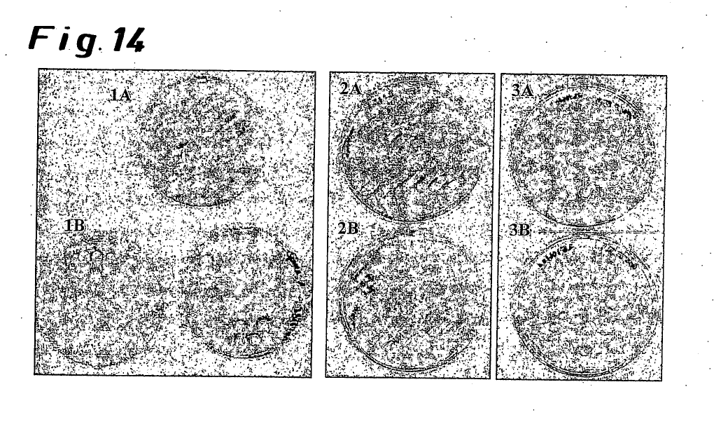

In order to generate a U. maydis-rpi knock-out strain, approx. 1.4 kbp genomic flanking regions in the 5′ and 3′ region of the rpi gene were amplified by a PCR reaction. After the flanks had been cut with SfiI, they were ligated with a hygromycin resistance cassette in order to construct a knock-out cassette. The resulting knock-out cassette was amplified in a PCR using nested primers. The PCR product was isolated and purified and then transformed into the diploid U. maydis strain FBD 11. The transformants obtained after selection on hygromycin were picked out. The knock-out strains were verified by PCR analysis and Southern Blot analysis. Two of the resulting strains were used to infect maize plants. After the resulting spores had germinated, they were verified for hygromycin resistance and for the presence of the wild-type gene (FIG. 14). No spores with deleted rpi gene were isolated.

### Example 2

Cloning, Expression and Purification of RPI1 or RPI1_{I from} Ustilago maydis

To express RPI1, the plasmid pRPI2 was transformed into E.coli BL21(DE3). A preculture was prepared by inoculating 5 ml of selection medium (dYT supplemented with 100 μg/ml ampicillin) with a single colony and incubating the mixture at 37° C. overnight in a shaker. The main culture (dYT supplemented with 100 μg/ml ampicillin) was inoculated at the rate of 1:80 and incubated at 37° C. with shaking; when an OD₆₀₀ of 0.7 had been reached, the culture was incubated for 1 hour at 18° C. and subsequently induced by addition of 1 mM IPTG (final concentration). After an incubation time of 21 hours at 18° C., the cells were harvested and subsequently frozen at −20° C. In this manner, the cells can be stored for several months at −20° C. without any loss in activity. 2.65 g of cells were suspended in 5 ml of binding buffer (50 mM potassium phosphate buffer, pH 8.0; 10% (v/v) glycerol, 300 mM NaCl), 750 μl of lysozyme (10 mg/ml) and 10 μl DNaseI (1 mg/ml) were added, and the mixture was incubated for 1 hour on ice. The cells were disrupted by 3 freeze-and-thaw cycles. After centrifugation at 20000 rpm (JA20) and 4° C. for 15 minutes, the supernatant was applied to an Ni-NTA column (volume: 1 ml) which had been equilibrated with binding buffer. The column was washed with 10 ml of binding buffer, and elution was effected at two imidazole levels of 40 mM (4 ml) and 200 mM (2 ml), RPI1 eluting at the 200 mM level. The protein fraction was subsequently transferred to storage buffer (50 mM Tris/HCl, pH8.0; 40 mM KCl, 10 mM MgCl₂) via a PD10 column. Approximately 3 mg of soluble RPI can be isolated from 200 ml of E. coli culture. Glycerol (final concentration 25%(v/v)) was added to the protein solution and the mixture was then frozen at −20° C. The enzyme which is isolated in this manner can be stored for several months at −20° C. without loss of an activity.

### Example 3

RPI Inhibition Tests for Finding Modulators

A) Spectrophotometric Method

To this end, the enzyme activity of ribose-phosphate isomerase is determined by the increase in absorption at 290 nm owing to the formation of ribulose-5-phosphate from ribose-5-phosphate. This assay may also be used for determining the K_{M} value and the activity of an RPI, for example the Ustilago maydis RPI 1.

The reaction volume amounted to 700 μl. Suitable volumes of a 50 mM ribose-5-phosphate stock solution were added to the assay buffer (0.1 M Tris/HCl, pH8.0, 0.5 mM DTT), and the reaction was started by addition of 1 μl of the RPI 1 preparation (see Example 1). The increase in absorption at a wavelength of 290 nm was monitored spectrophotometrically (Wood T., Assay for D-ribose-5-phosphate ketol isomerase and D-ribulose-5-phosphate 3-epimerase. Methods Enzymol. 41 (1975) 63-6; Wood, T., Spectrophotometric Assay for D-ribose-5-phosphate ketol isomerase and for D-ribulose-5-phosphate 3-epimerase. Analytical Biochemistry 33 (1970) 297-306). The change in absorption in the presence of a candidate compound at various concentrations was observed for comparison reasons. Reduced absorption suggests the presence of an RPI inhibitor.

B) Discontinuous Enzyme Assay with Carbazole and 75% (v/v) Sulphuric Acid

The assay is based on detecting the ribulose keto group by means of a colour reaction with a carbazole/cystein/HCl solution as purple dye (546 nm) (G. F. Domagk and K. M. Doering, Methods in Enzymology 41 (1975) 424ff, H. Horitsu, I. Sasaki, T. Kikuchi, H. Suzuki, M. Sumida and M. Tomoyeda. Purification, properties and structure of ribose 5-phosphate ketol isomerase from Candida utilis. Agr.Biol.Chem. 40(2) (1976) 257-264). The reaction mixture in a volume of 50 μl consists of 5-10 mM ribose-5-phosphate and 30 ng of RPI enzyme, which are incubated for 15 minutes at 37° C. 5 μl of a cystein solution (0.03 M) and 30 μl 75% (v/v) of sulphuric acid and 1 μl of ethanolic 0.1% (w/v) carbazole solution are then added to this mixture. The enzyme reaction is monitored with reference to a resulting coloration whose intensity increases with the amount of substrate converted (FIG. 12). When adding a candidate compound, an inhibitory effect of the compound can be identified on the basis of the decrease in absorption or simply the less pronounced coloration.

C) Coupled Enzyme Assay with ribulose-5-phosphate kinase. PK and LDH

In this case, an enzyme reaction is detected fluorometrically via the decrease in NADH (Che-Hun Jung, F. C. Hartman, Tse-Yuan, S. Lu, and Frank W. Larimer. D-ribose-5-phosphate isomerase from Spinach: heterologous overexpression, purification, characterization, and site-directed mutagenesis of the recombinant enzyme. Arch. Biochem. Biophys. 373 (2000) 409). Here, the conversion of interest of ribose-5-phosphate to ribulose-5-phosphate is coupled with further enzyme reactions which eventually leads to the decrease in NADH and thus to a decreae in the relative fluorescence in the course of the reaction (cf FIG. 13):

RPI

| | | |
|---|---|---|
| ribose-5-phosphate | → | ribulose-5-phosphate |

Phosphoribulose kinase

| | | |
|---|---|---|
| ribulose-5-phosphate + ATP | → | ribulose-1,5-diphosphate + ADP |

Pyruvate kinase

| | | |
|---|---|---|
| phosphenol pyruvate + ADP | → | ATP + pyruvate |

Lactate dehydrogenase

| | | |
|---|---|---|
| pyruvate + NADH + H⁺ | → | lactate + NAD⁺ |

When a candidate compound is added, no, or a reduced, decrease in relative fluorescence is observed when the tested compound brings about an inhibition of the RPI enzyme activity.

D) Coupled Enzyme Assay with ribulose-5-phosphate epimerase, transketolase, glycerinaldehyde-3-phosphate dehydrogenase

Ribose-5-phosphate isomerase converts ribose-5-phosphate into ribulose-5-phosphate, which is reacted further by D-ribulose-5-phosphate epimerase (EC 5.1.3.1) to give xylulose-5-phosphate. In a transketolase reaction (EC 2.2.1.1), the latter acts as donor ketone for the acceptor aldehyde ribose-5-phosphate, which is not added separately in this case since it already acts as substrate for the enzyme to be analyzed. Thus, only 0.5 mol of glycerinaldehyde-3-phosphate is obtained per mol of ribose-5-phosphate. Ribose-5-phosphate is reacted rapidly only when large amounts of ribose-5-phosphate isomerase are present. Glycerinaldehyde-3-phosphate is then reacted further by GAP-DH, NAD⁺being converted into NADH, so that the reaction of ribose-5-phosphate to ribulose-5-phsophate eventually leads to an increase in NADH.

The test substances are introduced into a 384-well microtitre plate in 5 μl of 5% (v/v) DMSO. The concentration of the substances is such that the final concentration of the substances in the assay carried out is 10 μM. 10 μl of enzyme solution (cooled at 4° C.) are pipetted thereto. The enzyme solution is composed as follows: 243 mM imidazole buffer (pH 7.6), 27 mM MgCl₂, 0.02% (w/v) cocarboxylase (TPP), 0.1% (w/v) BSA, 0.01 % (v/v) Tween 20, 90 mM NAD, 0.00038 u/μl yeast ribulose-5-phosphate epimerase (Sigma, Taufkirchen), 2.5 ng/μl isolated RPI1 (see Example 2). 20 μl of the substrate solution (cooled at 4° C.) are added to this mixture, thus starting the reaction. The substrate solution comprises 162 mM imidazole buffer (pH 7.6), 18 mM MgCl₂, 0.03 %(w/v) cocarboxylase (TPP), 0.075% (w/v) BSA, 0.0075% (v/v) Tween 20, 13.128 mM ribose-5-phosphate, 0.5625 ng/μl transketolase (maize transketolase heterologously expressed in E. coli), 0.00039 u/μl GAPDH (SIGMA, Taufkirchen), 10.8 mM NaH₂AsO₄. The increase in fluorescence at λ=360/35 nm (extinction) and λ=465/3 5 nm (emission) is measured for 3 hours at room temperature (determined in a preliminary measurement), the results of a measurement in the presence of a test compound being compared with the results of a measurement in the absence of a test compound. The above-described mixture in the absence of RPI1 acted as the control. The substances used in the assay were present in the following final concentrations: c(imidazole)=162 mM, c(MgCl₂)=18 mM, c(cocarboxylase)=0.03% (w/v), c(ribose-5-phosphate)=7.5 mM, c(NAD)=25.71 mM, c(NaH₂AsO₄)=6.17 mM, c(BSA)=0.071% (w/v), c(Tween-20)=0.007% (v/v), c(transketolase)=0.314 ng/μl, c(GAPDH)=0.00022 U/μl, c (ribulose-5-phosphate 3-epimerase)=0.000109 U/μl, m(RPI)=0.714 ng/μl.

### Example 4

Demonstration of the Fungicidal Effect of the RPI Inhibitors Identified

A methanolic solution of the active substance (Tab. I) identified with the aid of a method according to the invention, reacted with an emulsifier, was pipetted into the wells of microtitre plates. After the solvent had evaporated, 200 μl of potato dextrose medium were added to each well. Suitable concentrations of spores or mycelia of the test fungus (see Table II) were previously added to the medium.

The resulting emulsifier concentration was 300 ppm.

The plates were subsequently incubated on a shaker at a temperature of 22° C. until sufficient growth was observed in the untreated control. Evaluation was done photometrically at a wavelength of 620 nm. The dose of active compound which leads to a 50% inhibition of the fungal growth over the untreated control (ED₅₀) is calculated from the readings of the different concentrations (see Tab. II).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A method of identifying a fungicide, comprising
(a) bringing ribose-5-phosphate isomerase enzyme or a host cell comprising a polypeptide from phytopathogenic fungi encoded by a nucleic acid with the biological activity of ribose-5-phosphate isomerase into contact with a chemical compound or a mixture of chemical compounds under conditions which allow the interaction of the chemical compound or the mixture of chemical compounds with the ribose-5-phosphate isomerase enzyme or the polypeptide,
(b) comparing ribose-5-phosphate isomerase activity in the absence of the chemical compound or the mixture of chemical compounds with the ribose-5-phosphate isomerase activity in the presence of the chemical compound or the mixture of chemical compounds, and
(c) identifying the chemical compound or mixture of chemical compounds which specifically inhibit ribose-5-phosphate isomerase activity.

2. The method according to claim 1, fungicidal action of the identified chemical compound or mixture of chemical compounds is tested in a subsequent step (d) by bringing said identified chemical compound or mixture of chemical compounds into contact with a fungus.

3. A method of identifying one or more fungicidal compounds comprising identifying said fungicidal compound with a member selected from the group consisting of a polypeptide having the biological activity of ribose-5-phosphate isomerase, a nucleic acid encoding a polypeptide with the biological activity of ribose-5-phosphate isomerase and one or more host cells said host cell including a polypeptide having the biological activity of ribose-5-phosphate isomerase.

4. A fungicide, said fungicide comprising a modulator of a polypeptidehaving the biological activity of ribose-5-phosphate isomerase.

5. A method for controlling phytopathogenic fungi comprising controlling phytopathogenic fungi with a modulator of a polypeptide having the biological activity of ribose-5-phosphate isomerase.

6. A fungicidal modulator of a polypeptide having the biological activity of ribose-5-phosphate isomerase which modulator is identified by a method according to claim 1 or 2.

7. A nucleic acid encoding a polypeptide from phytopathogenic fungi with the biological activity of ribose-5-phosphate isomerase.

8. The nucleic acid as claimed in claim 7, wherein said nucleic acid encodes a U. maydis ribose-5-phosphate isomerase.

9. The nucleic acid according to claim 7 or 8, wherein said nucleic acid takes the form of single-stranded or double-stranded DNA or RNA or fragments of genomic DNA or cDNA.

10. The nucleic acid as claimed in claim 7, comprising a sequence selected from
a) the nucleic acid sequence of SEQ ID NO: 1,
b) a nucleic acid sequence which encodes a polypeptide with the amino acid sequence of SEQ ID NO: 2,
c) a nucleic acid sequence which encodes a polypeptide with at least one of the consensus sequences of the following group of consensus sequences:
-(I/V)GIGSGSTV-, -(I/V)D(I/V)X₂DGADE(I/V)DX₂LX₂IKG
G-, -(P)TG(F/D)QSX₂LI-, -EK(V/L)X₄AX₂F(I/V)XVADX
(R/S)K-, -WX₂G(I/V)PIEVXP-, -AKAGP(I/V)VTDNXNFX(I/
V/L)D-, -IKXLXGVXEXGLF-, -AYFGNXDG-,
d) part-sequences of the sequences defined under subparts a) to c) of this claim 10 which are at least 15 base pairs in length,
e) sequences which hybridize with the sequences defined under subparts a) to c) of this claim 10 at a hybridization temperature of 42-65° C.,
f) sequences with at least 60% identity with the sequences defined under subparts a) to c) of this claim 10,
g) sequences which are complementary to the sequences defined under subparts a) to f) of this claim 10, and
h) sequences which, owing to the degeneracy of the genetic code, encode the same amino acid sequence as the sequences defined under subparts a) to c) of this claim 10.

11. A DNA construct comprising a nucleic acid according to any one of claims 7 to 10 and a heterologous promoter.

12. A vector comprising a nucleic acid according to any one of claims 7 to 10, or a DNA construct according to claim 11.

13. A vector according to claim 12, wherein the nucleic acid is linked operably to regulatory sequences which ensure the expression of the nucleic acid in prokaryotic or eukaryotic cells.

14. A host cell comprising a member selected from the group consisting of a nucleic acid according to any one of claims 7 to 10, a DNA construst according to claim 11 and a vector according to claim 12 or 13.

15. A polypeptide from phytopathogenic fungi having the biological activity of ribose-5-phosphate isomerase, wherein said polypeptide is encoded by a nucleic acid according to any one of claims 7 to 10.

16. A method for finding a chemical compound or a mixture of chemical compounds according to claim 1 or 2, comprising identifying said chemical compound or mixture of chemical compounds with a polypeptide according to claim 15.

17. A method of finding a compound which modifies the expression of the polypeptide defined in claim 15, comprising:
(a) bringing a host cell according to claim 14 into contact with a chemical compound or a mixture of chemical compounds,
(b) determining a polypeptide concentration, and
(c) identifying the compound or mixture of compounds which influence the expression of the polypeptide.


SEQUENCE LISTING (2 sequences)

SEQ ID NO: 1 - DNA, 1020 bp, Ustilago maydis
  CDS (1)..(1017)
atgcaatccaggcttctctctctggtttcatcccatcttgtgtctaagcgctgtttcatcgctcgcagtgctgcccttgcgccgcttctccttcacccccaacgtctacgtctcacttgccctcgctccttttcttctcaacaatctggcccacgcaagatggcttcttccaacgctaccaacagtaccagtgccgccagtgctgccaacaccaactcgtctgctttcaagagtgccgaattggctgctttgagcggcgtcgaggctgctaagcgtgctgctgcgtacgctgccgttgacaaccacgtcaagccgcagcacgagatcattggtattggctcgggctcaacggtgccttatgttgttgagcgaattgctcaacagggacctgctgtgaatgcaaagcgttggttcgttcccaccggcttccagtcacgcgaactcatcatcaacgccggccttcgtctcggtgatgtggatagcttccccagcatcgacgtcactatcgatggcgctgacgaggtcgacaatgcgctcaactgcatcaagggcggcggtgcttgtcatttgcgcgaaaaggtactcgccgaagccgccaatgaatttgtcgtagtcgctgactaccgcaagaatggatcgcagctcggcacaaagtggctgcaaggtgtccccatcgaggtcgctccgtttgcctatgcaaaagtgcttcagaacctcaaaaagatgggttctgacaaggcggtccttcgcatgggcaaggcaaaagccggtcccgtcgttacagataatggcaacttttgcatcgatgctcccttccccgaagcacagatgaaggatccctctgatttgctcaagcgtatcaagttgctcaccggtgtacttgaggtcggtctgttttgcaacatttgcaagtccgcctactttggcaacgatgacggcaccatcaccatcaaaaccgccgccggagatgtgcaagagggcgtccactttgacgtctccaaggcgcctgcaacagcataa

SEQ ID NO: 2 - protein, 339 aa, Ustilago maydis
MQSRLLSLVSSHLVSKRCFIARSAALAPLLLHPQRLRLTCPRSFSSQQSGPRKMASSNATNSTSAASAANTNSSAFKSAELAALSGVEAAKRAAAYAAVDNHVKPQHEIIGIGSGSTVPYVVERIAQQGPAVNAKRWFVPTGFQSRELIINAGLRLGDVDSFPSIDVTIDGADEVDNALNCIKGGGACHLREKVLAEAANEFVVVADYRKNGSQLGTKWLQGVPIEVAPFAYAKVLQNLKKMGSDKAVLRMGKAKAGPVVTDNGNFCIDAPFPEAQMKDPSDLLKRIKLLTGVLEVGLFCNICKSAYFGNDDGTITIKTAAGDVQEGVHFDVSKAPATA